(19) United States
(12) Patent Application Publication
(10) Pub. No.: US 2005/0019776 A1
(43) Pub. Date: Jan. 27, 2005

(54) Universal selective genome amplification and universal genotyping system

(76) Inventors: Matthew James Callow, San Mateo, CA (US); Radoje T. Drmanac, Palo Alto, CA (US); Snezana Drmanac, Palo Alto, CA (US)

Correspondence Address: Renee S. Polizotto, Ph.D.;NUVELO, INC., 675 Almanor Avenue, Sunnyvale, CA, 94085, US

(21) Appl. No.: 10/608,293

(22) Filed: Jun. 27, 2003

Related U.S. Application Data
(60) Provisional application No. 60/392,625, filed on Jun. 28, 2002.

(51) Int. Cl. C12Q 1/68; C12P 19/34

(52) U.S. Cl. 435/6; 435/91.2

(57) ABSTRACT

The invention relates to methods for isolating and amplifying small fragments of genomic DNA for genotyping polymorphisms in human populations.

### CROSS REFERENCE TO OTHER APPLICATIONS

This application claims the benefit of U.S. Provisional Application Ser. No. 60/392,625 filed on Jun. 28, 2002, entitled “Universal Selective Genome Amplification and Universal Genotyping System,” Attorney Docket No. CAL-1. This and all other U.S. Patents and Patent Applications cited herein are hereby incorporated by reference in their entirety.

### 1. TECHNICAL FIELD

The invention relates to methods for isolating and amplifying small fragments of genomic DNA for genotyping polymorphisms in human populations. In certain aspects of the invention, the methods are useful in performing nucleic acid sequence analysis.

### 2. BACKGROUND

Humans share 99.9% genomic sequence identity; therefore, variations at sites representing the remaining 0.1% are responsible for the genetic variation between individuals, including the differences in risk for diseases and response to drugs. Technologies that enable an association to be made between these specific sites of inherited sequence variation, called single nucleotide polymorphisms (SNPs) and disease traits have a great potential for treatment and/or providing cures for these diseases.

There is an ever increasing demand for rapid and accurate, but also economical technologies to genotype polymorphisms in human populations. Many of the existing approaches to detecting known polymorphisms rely upon the custom generation of reagents specific for each polymorphism, which can be costly and time consuming. PCR amplification has been used for detection of SNPs by utilizing the 3′-match/mismatch feature of an annealing primer for selective amplification (Newton, et al., Nucleic Acids Res. 17:2503-2516 (1989), herein incorporated by reference). In a similar way, the oligonucleotide ligation assay relies upon the ligation of one or two allele-specific probes to an adjacent fluorescent probe when hybridized to a PCR-amplified gene fragment (Mahe and Corthier, Can. J. Microbiol. 34:916-918 (1988), herein incorporated by reference). Only when there is a perfect match between the variant or wild-type probe and the PCR-amplified DNA will the ligation occur which can be detected by electrophoresis.

Rolling circle amplification (RCA) has been used in which two allele-specific probes of about 90 bases in length are synthesized. Both probes contain sequence complementary to the sequence surrounding the polymorphic site, but each probe contains a different 3′ base. Ligation of the probe ends to form a circle is dependent upon the specific base identity at the polymorphic site. RCA of each circular probe can then occur utilizing primer binding sites in the backbone of the probe (Banér, et al., Nucleic Acids Res. 26:5073-5078 (1998); Nallur, et al., Nucleic Acids Res. 29:E118 (2001), both herein incorporated by reference).

Fluorescence Resonance Energy Transfer (FRET) has been utilized with molecular beacons in which donor-acceptor dye pairs are attached to each end of complementary sequences flanking the target-specific sequence. When the target is not hybridized, the probe maintains a hairpin conformation resulting in donor fluorescence quenching, however when hybridized to the correct target, donor and quencher are separated resulting in fluorescence emission (Tyagi, et al., Nat. Biotechnol. 16:49-53 (1998), herein incorporated by reference). Several other strategies for SNP detection utilize FRET such as in the TaqMan™ assay (Livak, et al., PCR Methods Appl. 4:357-362 (1995)) and dye-labeled oligonucleotide ligation (Shuber, et al., Hum. Mol. Genet. 6:337-347 (1997), all herein incorporated by reference).

Microarrays have also been applied to the detection of SNPs through the synthesis of allele-specific probes in the form of high-density arrays and their hybridization to fluorescently-labeled, PCR-amplified DNA (Chee, et al., Science 274:610-614 (1996), herein incorporated by reference). A limitation with this approach is the need to perform many PCR reactions for each array. An alternative approach has been to screen a few SNPs from many individuals by arraying patient samples on a solid support (Shuber, et al., Hum. Mol. Genet. 6:337-347 (1997), herein incorporated by reference).

In dynamic allele-specific hybridization (DASH), PCR is used to amplify a product with one biotinylated primer that allows capture of single stranded DNA onto the base of a well. A duplex-binding fluorescent dye is then used to measure the release temperature of a hybridized probe. Mismatches caused by polymorphisms can be detected because of the separation of the duplex (and dye) at a lower temperature than for a full-match (Forster, et al., Arch. Neurol. 32:54-56 (1975), herein incorporated by reference).

Type IIS endonucleases, or “outside cutters,” are restriction enzymes that produce a cut outside of the recognition sequence (Roberts and Macelis, Nucleic Acids Res. 29:268-269 (2001), herein incorporated by reference). The use of Type IIS restriction endonucleases to fragment DNA and the capture of those fragments by ligation to adapters has been described (Smith, PCR Methods Appl. 2:21-27 (1992); Unrau and Deugau, Gene 145:163-169 (1994); both of which are herein incorporated by reference). These studies relied upon the incorporation of primer binding sites in the adapters to amplify the captured sequences over other sequences by standard PCR methodologies. Sibson and Gibbs (Nucl. Acids Res. 29:E95 (2001), herein incorporated by reference) recently described an approach to produce fragments from human genomic DNA using Type IIS restriction endonuclease digestion followed by sorting or indexing those fragments using successive rounds of digestion and ligation to adapters. The incorporation of Type IIS recognition sequences into adapters for cutting into adjacent sequences has also been applied in the technique of Serial Analysis of Gene Expression (SAGE) (Gunnersen, et al., Mol. Cell Neurosci. 19:560-573 (2002), herein incorporated by reference).

An improvement in genotyping polymorphisms and sequencing selected genomic fragments using universal adapters that allow for a high level of specificity would greatly facilitate analysis of medically important gene variants and sequencing of genomic fragments. Such an improvement would also eliminate the enormous cost of making and handling millions of pairs of SNP-specific primers or probes. Thus, there remains a need for additional and improved methods and materials for isolation, amplification, or sequencing genomic fragments and to genotype SNPs or other polymorphisms or mutations.

### 3. SUMMARY OF THE INVENTION

The method of the present invention reacts DNA from patient samples with two Type IIS restriction enzymes that fragment the genomic DNA into approximately 16 million fragments of 100 to 250 bp each that can be captured and amplified. The use of different enzyme types and combinations thereof result in the production of approximately 1 million to 50 million fragments. The output of this reaction is divided among the wells of a microtiter plate. In each of those wells, a plurality of different adapters is added, which will circularize the SNP-containing fragments and enable amplification of a plurality of up to about 3, 5, 10, 20, 50, 100, 200, 400, 500, 1000, or 2000 different SNPs in each well. Non-circularized fragments can be eliminated by digestion with exonuclease or removed by other means. The method of the invention further enriches the SNPs in each well by a second round of selection with another set of multiple adapters.

The present invention provides novel methods for providing low-cost, highly multiplexed single nucleotide polymorphism (SNP) diagnostics (see FIG. 1). The present invention also provides for a new system for using Nanobarcodes™ (NBCs) particles to provide a thousand or more distinct tags to allow full exploitation of the multiplexing capabilities of a sensitive and accurate SNP detecting chemistry. The present invention provides a method to score approximately 144,000 different SNPs from a miniscule volume of a single patient sample and can be scaled to test from hundreds to millions of SNPs. The method of the invention comprises two basic steps: amplification of SNP-containing DNA fragments, and capture of these sequences by NBCs that are subsequently detected by a detector that scores the SNPs.

The method of the invention provides for further processing of each reaction within the same well by adding a plurality of NBCs (2 for each biallelic SNP), each with a 6-mer probe attached and fluorescently labeled 5-mer probes in solution. The addition of the ligase enzyme will result in the ligation of the 6-base attached probe with the 5-base fluorescent probe when both 6- and 5-base complementary sequences are adjacent to each other in the target. Each amplified SNP will therefore generate a fluorescent signal on each NBC for which the matching SNP sequence is present. The present invention further provides for a 3-probe ligation to increase sequence specificity. The labeled probe will be selected to match after an unlabeled or labeled internal spacer probe ligates to the immobilized probe. The mixtures of the fluorescent NBCs are decoded and oligonucleotide binding is quantified.

Another embodiment of the method of the invention uses combinatorial ligation of three or more probe sets that may be represented by pools of probes to score large numbers of complete sets of probes longer than 10, 11, 12, 13, 14, 15, 16, 17, 18, 19, 20, 21, 22, 23, or 24 bases. The first set of probes (or probe pools) may be attached to a support, and the last set of probes (or probe pools) is labeled. Internal probe (or probe sets) may be unlabeled or labeled, wherein the labeled set of probes may represent donor or acceptor for FRET type of detection of the presence of all probes in the ligation construct.

Another embodiment of the method of the present invention is for diagnostic SNP testing. It is possible using the universal probe technology to design the process to amplify and genotype any particular subset of SNPs.

The method of invention also provides methods for preparation of adapter and other DNA from universal building blocks with complementary sequences. In one example, two sets of 256 DNA fragments all with different 4-base overhang sequences on one end, and longer overhangs complementary between sets allow preparation by ligation of about 32,000 double sided adapters. In another embodiment, adapters or any other double stranded DNA including full length genes is prepared by stepwise ligation of overlapped short (5-mers to 8-mers) oligonucleotides selected from one or more universal sets containing 50% or more, 75%, or more 90% or more, or all possible oligonucleotide sequences of given length.

The method of the present invention provides for various types of universal adapters: single-sided, double-sided, 2-, 3-, 4-, 5-, 6-, or 7-base long sticky ends, as well as universal sets of mixtures of adapters with different sticky ends, containing one or more restriction and primer sites. The invention further provides various methods for selective isolation or amplification of individual genomic fragments or mixtures of fragments.

The invention also provides a method of amplifying genomic fragments comprising the steps of: a) digesting genomic DNA into fragments, wherein said digesting results in genomic fragment overhangs; b) contacting said genomic fragments with one or more adapters, wherein said adapters are complementary to at least some of said overhangs; c) ligating said adapters to said genomic fragment overhangs to form closed adaptor-genomic fragment circles; d) separating said adapter-genomic fragment circles from linear fragments; and e) amplifying said adapter-genomic fragment circles.

The invention also provides a method of amplifying genomic fragments comprising the steps of: a) digesting genomic DNA into fragments, wherein said digesting results in genomic fragment overhangs; b) contacting said genomic fragments with one or more adapters, wherein said adapters are complementary to at least some of said overhangs; c) ligating said adapters to said genomic fragment overhangs to form closed adapter-genomic fragment circles; d) modifying said circles by digesting with restriction enzymes that recognize one or more adapter site; and e) amplifying said adapter-genomic fragment circles.

The invention also provides a method of amplifying genomic fragments comprising the steps of: a) digesting genomic DNA into fragments, wherein said digesting results in genomic fragment overhangs; b) contacting said genomic fragments with a set of universal adapters, wherein said adapters are complementary to at least some of said overhangs; c) ligating said adapters to said genomic fragment overhangs to form closed adapter-genomic fragment circles; d) separating said adapter-genomic fragment circles from linear fragments using an exonuclease to digest said linear fragments, e) amplifying said adapter-genomic fragment circles; and f) removing said genomic fragments from the adapter-genomic fragment circles.

The preferred embodiment of the invention uses Type IIS restriction endonucleases selected from the group consisting of Bbv I, SfaN I, Fok I, BsmF I, and BsmA I, wherein the restriction enzyme recognition site is distinct from the cleavage site. According to the method of the present invention, Type IIS restriction enzymes recognize a 5-base recognition sequence and cleave at a different site, leaving a 4-base overhang. The method of the invention also uses a set of 256 universal adapters. Amplification of the adapter-genomic fragment circles is performed by PCR, such as rolling circle PCR or inverse PCR.

One embodiment of the method of the present invention provides for an adapter constructed such that two Type IIS restriction sites are incorporated into said adapter, wherein the first site is positioned to cut into the adjacent genomic sequence to form an overhang sequence generated from the genomic sequence, and the second site is positioned to cut into the adapter to form an overhang sequence that is complementary to the genomic sequence generated by the first site.

The method of the invention also provides for the incorporation of uracil into the adapter sequence such that the adapter will be cleaved at the point of incorporation by uracil-DNA glycosylase and heating in order to linearize the adapter prior to amplification using Taq or Vent DNA polymerase.

In addition, the method of the present invention provides for using biotinylated blocking adapters rather than exonuclease for removal of non-circularized fragments. The biotinylated adapters consist of all possible combination of overhangs for a given length equal to overhangs in the genomic fragments. One end of the biotinylated adapter has an overhang and the second end is bound to biotin in order to achieve removal with streptavidin-coated beads that can be collected by magnetic attraction or centrifugation.

The method of the invention creates a closed circular arrangement of nucleic acids comprising a plurality of genomic fragments. Such genomic fragments contain overhangs occurring at each end of said fragment and are complementary to a plurality of universal adapters. Such fragment overhangs are ligated to said adapters to form a closed circular arrangement of nucleic acids.

The method of the invention provides for preservation of a selected target DNA segment in a DNA mixture by ligating two blocked adapters or circularization by one double-sided adapter with matching sticky ends.

The method of the invention provides for tagging selected fragments with adapters in cycles of ligation with a specific adapter containing a second restriction enzyme recognition sequence. The second digest exposes new sequences in the target DNA fragment and is carried out using either single- or double-sided adapters.

The method of the invention provides for adapters that comprise one or two pairs of Type IIS restriction endonuclease recognition sites, each pair positioned at one adapter end to enable cutting into the genomic DNA and into the adapter DNA to form matching overhang sequences for selected genomic fragments.

The method of the invention utilizes universal adapters of two types. Single-sided adapters which have one end blocked and one end complementary to the genomic fragment sticky end. Double-sided universal adapters are sticky at both ends and can thereby circularize upon ligation with a genomic fragments.

One embodiment of the invention provides for multiple cycles of digestion and ligation at the same site to reduce mismatch background. Mismatch ligation products can also be removed by using mismatch or single-stranded DNA recognition enzymes.

The method of the invention provides for building universal adapters from at least one set of universal building blocks. The set of universal adapters will contain more than 256 members. The universal adapters may contain restriction recognition sites, primer sites, and/or internal informative sequences 2-6 bases in length that can be exposed by restriction enzyme cutting.

Adapters may be pre-assembled or building blocks can be annealed or annealed and ligated after mixing with genomic DNA fragments. For direct use of adapter building blocks, each building block can be prepared with 2-10 different assembly ssDNA tails corresponding to tails of 2-10 different core adapters to allow selection of specific building blocks that minimize formation of additional adapters by combinatorial ligation of all building blocks added in one reaction. To generate 8 specific adapters after mixing with genomic DNA, 56 others (8×8-8) can be generated if each building block for one end has the same tail. If four different tails are used, then a maximum of 8 [4×(2×2-2)] unwanted adapters will be created. To generate 16 adapters using 4 different tails, a maximum of 48 [4(4×4-4)] instead of 240 unwanted adapters may be formed.

The method of the invention provides for methods to prepare pools of fragments with SNPs which a) consist of any sequence, and b) share N-mers close to the polymorphic site.

One embodiment of the invention reduces the complexity of the genomic fragment mixture by digesting with restriction enzymes that produce blunt ends or sticky ends with improper lengths.

Another embodiment of the invention provides for consecutive ligation, cutting, and ligation of mixed adapters with the proper sequence and length of sticky ends and restriction enzyme sites.

### 4. DESCRIPTION OF THE DRAWINGS

FIG. 1 depicts a schematic portraying NBC synthesis and whole-genome SNP screening of 144,000 SNPs per sample and diagnostic SNP testing of 1000 disease-linked SNPs.

FIG. 2 depicts fringe scanning (Df=2λsin(β/2)), adapted from Mullikin et al., Cytometry 9:111-120 (1988).

FIG. 3 shows slit and fringe illumination profiles and frequency responses, adapted from Mullikin et al. 1988 supra, wherein 3a and b depict the scanning beam and Fourier transform, respectively, for a slit-scan beam with a full width at 1/e² points of 1.5 μm; 3c and d depict the scanning beam and Fourier transform, respectively, for a fringe-scan field with a fringe spacing of 1.05 μm; 3e and f depict the scanning beam and Fourier transform, respectively, for a fringe-scan field with a fringe spacing of 0.52 μm.

FIG. 4 shows the Bbv I recognition sequence (GCAGC) and the cut 8 bases downstream leaving a 4-base, 5-prime overhang.

FIG. 5 illustrates strategies for adapter synthesis, wherein A represents annealing of complementary sequences in the 2 oligonucleotides resulting in the formation of an adapter with 4-base overhangs at each end; B represents successive annealing of 7-mer oligonucleotides used to generate the desired adapter; C represents 8-mer oligonucleotides annealed to a 60 bp core sequence cut from a plasmid; thin lines represent complementary single-stranded sequences.

FIG. 6 shows the recognition sites in the adapter and the genomic sequences when using the internal cut adapter strategy.

FIG. 7 illustrates examples of possible ligation events that could occur, wherein A, B, and C represent unique 4-mer overhangs; A′, B′, and C′ represent the complementary overhangs to A, B, and C, respectively; thin lines represent the adapter DNA; thick lines represent the genomic fragment DNA.

FIG. 8 shows the recognition sequences engineered into the primary adapter to allow outside cutting into the genomic sequence captured in the primary ligation event, wherein the solid lines represent the adapter and the hatched lines represent the genomic sequence.

FIG. 9 depicts a 3-piece ligation strategy. Allele A is a perfect full-match to the sample sequence and therefore is subject to ligation to the internal probe followed by ligation to the labeling probe. Allele G is mismatched and is not ligated with the internal labeling probe. Both NBCs are scored and analyzed to determine which probe is complementary to the sample sequence.

FIG. 10A shows a gel demonstrating the isolation of genomic DNA fragments from E. coli with a single round of adapter selection and amplification, wherein lane 1 contains the 50 bp marker, lane 2 contains the 100 bp fragment, lane 3 contains the 150 bp fragment, lane 4 contains the 200 bp fragment, lane 5 contains a mix of 3 adapters, one for each fragment, and lane 6 contains the 50 bp marker.

FIG. 10B shows a gel demonstrating the isolation of human genomic DNA fragments from the second round of adapter selection and amplification, wherein lane 1 contains the 50 bp marker, lane 2 contains the 125 bp fragment, lane 3 contains the 262 bp fragment, lane 4 contains the 318 bp fragment, lane 5 contains the 499 bp fragment, and lane 6 contains fragments from a multiplex of 125, 262, 318, and 499 bp adapters.

FIG. 11 illustrates an adapter with 4-base overhangs, 2 primer binding sites and an outside cutter recognition site.

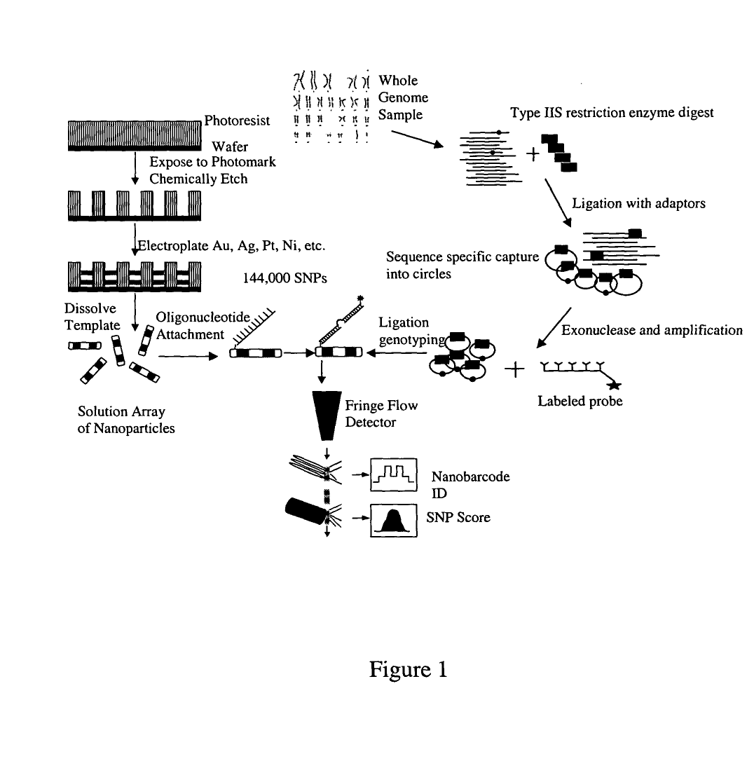

### 5. DETAILED DESCRIPTION OF THE PREFERRED EMBODIMENTS

5.1 Nanobarcodestm Particles

NBC identification tags, developed by Natan and colleagues (PCT International Publication No. WO 01/25002 to Natan and Mallouk; Nicewarner-Pe\ddot{n}a et al., Science 294:137-141 (2001), herein incorporated by reference) are nanoscale metallic rods with thousands of unique patterns of metal segments along their length. NBCs are cylindrical in shape with metallic composition varying along the length of the rod and typically have diameters of tens to hundreds of nanometers, and lengths of 1-10 μm. Because the width and composition of each segment can be varied, the nanobar is designed to act as a “barcode” on the nanometer scale. Although the diameter of the nanobars is usually of nanometer scale, the overall length can be chosen so that the barcode stripe pattern can be visualized directly in an optical microscope or other optical system, exploiting the differential reflectivity of the metal components. Due to their small size and the ability to coat the metal surfaces with many different types of molecules, NBCs are well suited to provide a foundation for highly multiplexed solution-phase assays (solution arrays).

NBCs with 4 or 9 stripes, each of a different metal (e.g. gold (Au), silver (Ag), palladium (Pd), platinum (Pt), nickel (Ni), and copper (Cu)) with a different reflectivity, are available. Thus, there are more than 130,000 unique types of coded metal nanoparticles that could be prepared. This number is less than 4⁹ (=262,144) by about a factor of two because palindrome designs are not covered. Thiol-terminated molecules, such as DNA probes, can be directly attached to NBCs. The efficiency of derivatization can be monitored by hybridization to complementary oligonucleotides containing fluorescent tags.

In a particle-based assay system, the presence of an analyte is detected by its binding to the substrate particle and a fluorescent label. The detection system must quantitate the bound fluorescence and detect the substrate used to bind the analyte. An example of substrate identification includes detection of multiple fluorescent colors and bead size with an optical microscope or flow analyzer. In the case of NBCs, identity is determined by decoding the striped pattern by light microscopy.

Features as small as 60 nm can be identified; however, the current working minimum stripe width for accurate identification is above twice the Rayleigh criterion (the minimum resolvable detail between two wavelengths) (Barenghi, Phys. Rev. B. Condens. Matter, 52:3596-3600 (1995), herein incorporated by reference). A change in reflectivity at a particular wavelength can be exploited to identify multiple metals. Contrast between silver and gold is maximized below 488 nm, but disappears above 600 nm. Palladium and platinum are continually dimmer than silver but become dimmer than gold above 500 nm. The NBC system (SurroMed, Mountain View, Calif.) takes images at these two wavelengths in order to distinguish three or more metals (e.g. gold, silver, and palladium) using an automated inverted microscope to identify the particles and measure fluorescence.

### 5.1.1 Nanobar™ Manufacturing and Multiplexing

The current nanobar synthesis method uses commercially available porous alumina membranes that are inexpensive, easy to plate with metal, and readily available. They have nominal 200 nm pore diameter and greater than 1 billion pores per 1-inch diameter disc. To eliminate problems of pore diameter variation, quality control, and occasional branching of pores, novel electroforming templates using photolithography with a much higher pore count will be used (see SurroMed and Callida Genomics NIST-ATP-00-01 grant application).

Photolithographically-produced pores provide greater diameter control and high pore density and are manufactured in a well-established manufacturing process. In addition, methods for electroplating in photoresist are well established (Romankiw et al., Handbook of Microlithography, Micromachining and Microfabrication Vol. 2, SPIE Press, 197 (1997) “Plating Techniques”, herein incorporated by reference). Conventional lithography can produce 300 nm diameter pores. However, direct exposure produces only 6:1 aspect ratios at this minimum diameter (the physical length of the vertical axis divided by that of the horizontal axis). Significantly higher aspect ratios are preferred in order to obtain nanobars with sufficient length to contain complex striping patterns, but narrow enough to have good flow characteristics in a flow-based detector. Such high aspect ratios will be obtained using dry reactive ion etching (RIE) of polymers. RIE is simple, flexible and controllable. One-micron features with aspect ratios of up to 32:1 have been demonstrated (Romankiw et al., 1997. supra) by single step RIE.

Initially simple single- or dual-metal nanobar designs are synthesized and the reproducibility of their manufacture within and across template membranes is characterized. Scanning electron microscopy is an efficient technique for measuring nanobar diameters and lengths, and is used extensively.

The nanobar striping patterns are based on the differential reflectivity of metals at a given wavelength. Table 1 lists the number of types that may be prepared as a function of both number of stripes and number of metals used. For example, to achieve 1,000 flavors, nanobars are made with 3 metals and 7 stripes. Using 8 stripes will yield a larger margin of error if some patterns cannot be identified. At 430 nm gold, silver, and palladium have quite different reflectivity and are the preferred choices for NBC synthesis. The use of four or more metals would be beneficial, but more difficult with sufficient contrast.

TABLE 1
NBC patterns available versus # of stripes and # of metals.
# of patterns = (mⁿ + m^{ceil(n/2)})/2;
m = #of metals, n = # of stripes

| # of | | | | |
|---|---|---|---|---|
| Stripes | 2 Metals | 3 Metals | 4 Metals | 5 Metals |
| 1 | 2 | 3 | 4 | 5 |
| 2 | 3 | 6 | 10 | 15 |
| 3 | 6 | 18 | 40 | 75 |
| 4 | 10 | 45 | 136 | 325 |
| 5 | 20 | 135 | 544 | 1625 |
| 6 | 36 | 378 | 2,080 | 7,875 |
| 7 | 72 | 1,134 | 8,320 | 39,375 |
| 8 | 136 | 3,321 | 32,896 | 195,625 |
| 9 | 272 | 9,963 | 131,584 | 978,125 |
| 10 | 528 | 29,649 | 524,800 | 4,884,375 |
| 11 | 1,056 | 88,938 | 2,099,200 | 24,421,875 |

The overall nanobar length will depend on the minimum stripe length that can be reliably detected. Flow detection equipment will have a resolution of about 1 micron, resulting in nanobars measuring 7 μm in length. Should longer stripes be required, the NBC length must increase accordingly. The choice of membrane templates controls rod diameter, the optimum of which is determined by two competing interests: wider rods result in a larger fluorescence signal, but narrow rods are more easily aligned in the flow stream.

It is possible that achieving particle suspension or consistent particle alignment in the flow detector may place an upper limit on nanobar diameters that is smaller than the minimum pore size that can be obtained by photolithography. In this case, two alternative template designs will be investigated. One alternative is the use of a SiC stamp to initiate the anodization process for the formation of pores in anodized aluminum (Masuda et al., Appl. Phys. Lett. 71:2770-2772 (1997), herein incorporated by reference). This method results in extremely uniform pores with diameters from 10 nm to 120 nm and high aspect ratios. Another possibility is the patterning of submicron pores in photoresist using interference lithography (Savas et al., J. Appl. Phys. 85:6160 (1999), herein incorporated by reference). With both of these techniques, an indirect template synthesis method could be used to avoid difficult or expensive template synthesis. The original template can be used to form a replication template that is an array of rods, which may then be used to mold a polymer such as polymethylmethacrylate (PMMA). The polymer molds can then be used for the formation of nanobars.

Although nanobar manufacturing methods may result in pore diameter or length variations that are greater than can be tolerated for accurate identification or signal quantitation, fluorescence quantitation accuracy will likely be adequate. Because plating thickness variation across large surface areas have been encountered in LIGA micromachining projects, the electroplating process can be improved to minimize any length variations that may limit accurate classification each flavor of nanobar. Alternatively, the number of wells produced by the universal adapter process may be increased (and the number of SNPs per well decreased), or the target of 144,000 SNPs per sample may be reduced to a lower number.

Stability studies such as the assessment of storage conditions and surface treatments will be conducted to improve shelf life. Oxidation of the nanobar surface during storage may change the density of oligonucleotide attachment or reflectivity profiles, thereby reducing the shelf life.

### 5.1.2 Oligonucleotide Attachment Chemistry

The present invention provides approaches for generating an array of nanobars, each member containing a unique identifying sequence to enable multiplexed SNP analysis (Shoemaker et al., Nat. Gen. 14:367 (1996), herein incorporated by reference). The oligonucleotides are coupled to the nanobars in a robust manner that allows efficient hybridization of the target sequences. Additionally, the coupled oligonucleotides are compatible with hybridization buffers and elevated temperatures (94, 95, 96, 97, or 98° C.) for denaturation.

Mirkin and coworkers (Storhoff et al., J. Am. Chem. Soc. 120:1959-1964 (1998), herein incorporated by reference) have shown that thiol-terminated oligonucleotides can be directly attached to nanoparticles of gold. An alternate, flexible architecture for oligonucleotide attachment to NBC has two parts: a common spacer with nanobar attachment chemistry and a common nucleic acid. With this approach the HS-Spacer is first attached to the nanobar as a self-assembling monolayer (SAM). The same can contain a functional group for linking to the DNA. A common form for the SAM is HS—(CH₂)ₙCOOH, where n is 12, 13, 14, 15, 16, 17, or 18. Longer-chain thiols form films that are thermally more stable than films formed from short-chain thiols (Bain and Whitesides, J. Am. Chem. Soc. 111:7164 (1989), herein incorporated by reference). Amine labeled oligonucleotides can be coupled to the carboxylate groups on the SAM with common conjugation (1-ethyl-3-(3-dimethylaminopropyl) carbodimide/N-hydroxysuccinimide (EDAC/NHS)) chemistry. The 5′ end of the oligonucleotide is near the nanobar. Alternative spacers, attachment chemistries, linker chemistries (sulfonates, guanindienes, carbodiimides, aldehydes, hydrazines and succinimidyl esters) and common NA sequences may be evaluated if problems arise with non-specific adsorption between DNA and the metallic surfaces or hybridization. Crosslinking may be used to help stabilize the SAM monolayer for higher temperatures.

The following specifications are set as the requirement for performance: 1) achievement of a hybridization oligonucleotide density of 10⁵ per μm²; 2) homogeneity of surface activation of <20% based on coverage per μm²; 3) particle-to-particle variability of <20%; 4) batch-to-batch variability of <20%; 5) attachment chemistry which shows no sequence dependency (i.e. <20% variation between coverage per μm² for each sequence); and 6) stability of attachment chemistry: <10% loss of attached oligonucleotide when incubated at 70° C. for 1 hour, preferably 94, 95, 96, 97, or 98° C. for 1 hour.

Another step may be added to further stabilize the linker chemistry the temperature ranges required for hybridization reactions, which requires cross-linking the SAM molecules to prevent desorption at high temperatures. Additionally, the effect of linking of oligos to the nanobars on the interaction of the particles with the hydrodynamic flow forces in the fringe-scan detector is monitored and flow conditions adjusted if necessary.

5.1.3 Nanobarcodes™ Detection

The present invention provides a prototype high speed reader that can identify and assay 128,000 SNPs in less than one hour. The instrument is a flow analyzer designed to be able to resolve one micron features for nanobar identification by reflectivity and measure up to four channels of laser induced fluorescence. Acquired reflectance and fluorescence signals is output to a custom software application that identifies each particle, measures bound fluorescence and tabulates the raw data for SNP analysis.

A particle flow analyzer provides the preferred solution for a high speed nanobar analyzer. Fluorescence assays of particles and cells in flow are a well-established technology (Shapiro, Practical Flow Cytometry, Wiley-Liss, New York, 1995, herein incorporated by reference). Modern multiparameter flow analyzers can process fluorophore-labeled cells at rates greater than 100,000 particles per second with a detection limit of a few hundred bound fluorophores per particle. For example, if 100 particles must be detected per flavor at 10,000 per second, the total analysis time for genome-wide analysis with 128,000 SNPs would be about 21 minutes. Flow analyzers typically do not image the objects they analyze. However, slit-scan and fringe-scan detection techniques have been used to resolve single micron features (Bartholdi, et al., Cytometry 10:124-133 (1989); Mullikin et al., Cytometry 9:111-120 (1988), both herein incorporated by reference). Slit and fringe techniques are used to generate sub-micron nanobar reflectivity profiles to enable high-speed identification of nanobars.

The alignment of long particles in flow analyzers has been demonstrated for chromosomes (Melamed et al., Flow Cytometry and Sorting, 2^{nd} ed. Wiley-Liss, p. 27 (1990), herein incorporated by reference). The laminar flow of the sample in tubing has a parabolic velocity profile (Shapiro, 1995. supra), i.e. zero velocity at the tube walls and maximum velocity in the center. A long object oriented across the flow stream experiences a torque that aligns the particle's long axis to the flow. If the flow system is stable then the rods will remain aligned when the sample and sheath streams are combined and focused in the nozzle.

In slit scan techniques where the illumination is focused to a line, or conversely the collected signal is focused through a slit aperture, resolution limits of almost 1 micron have been achieved (Bartholdi, et al., 1989. supra). However, fine resolution results in reduced depth of focus and a limit in achievable resolution exists (see Table 2). The current microscope system employs 1.4 NA optics and thus can accurately identify NBCs with 0.4 to 0.5 micron stripes, just above the spot diameter, but the depth of focus is only 170 nanometers. With the same criteria it is extrapolated that a 0.4 NA lens could resolve down to 1.5 micron stripes with a depth of focus of plus or minus two microns. For a 7 micron long, 7 stripe nanobar library, a resolution of one micron or better is required. A typical free stream analyzer sample core can confine particles to approximately a five micron region; therefore for an analyzer to work for a large nanobar library tighter confinement or improved depth of focus is required.

TABLE 2

| | | Diameter of spot | Depth of focus |
|---|---|---|---|
| NA | Lambda | (∝ NA) | (∝ 1/NA²) |
| 1.40 | 0.488 | 0.42 | 0.17 |
| 0.85 | 0.488 | 0.69 | 0.45 |
| 0.50 | 0.488 | 1.17 | 1.30 |
| 0.40 | 0.488 | 1.46 | 2.03 |

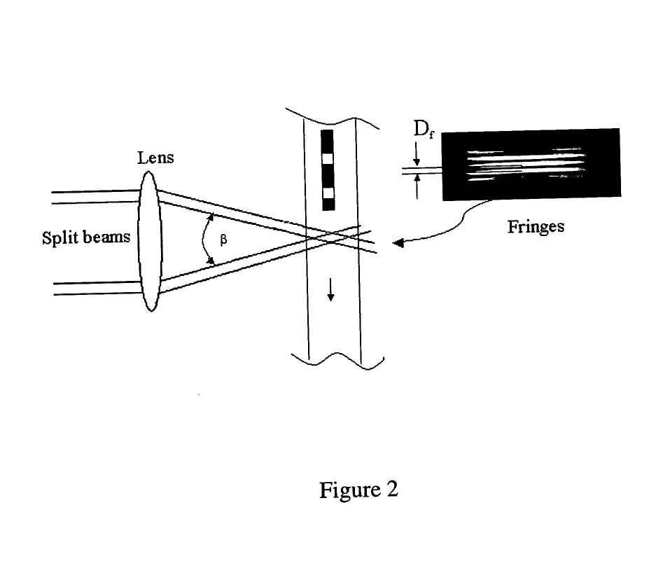

Fringe scan flow (Mullikin et al., 1988. supra) was developed to improve the spatial resolution of a flow system without sacrificing depth of focus. In fringe-scan flow, a single laser beam is split into two equal power beams that are then focused into the flow stream (see FIG. 2). The two beams interfere and create a fringe pattern that is proportional to the wavelength used and the focusing angle. The measured signal represents a convolution between the fringe pattern and the NBC striping pattern and is removed by a deconvolution. A nominal doubling of depth of focus and resolution of 0.7 microns has been reported.

The SurroMed flow analyzer will be developed to adapt the InFlux™ analyzer for slit and fringe scanning for construction of the first prototype systems, followed by the design of the optics, mechanics and electronics for slit and fringe illumination.

The instrument has two lasers, an Argon ion and HeNe (helium-neon) laser to generate light used for slit and fringe scanning, 488 nm and 633 nm respectively. The slit scan illumination is also used for laser induced fluorescence. The fringe-scan beams cross the flow stream at the focal point of the lens generating an interference pattern. Fringe scanning occurs as the objects pass through the interference pattern (see FIG. 2). The fringe-scan beams are also spatially separated downstream from the slit scan beam. The resulting side scattered light that occurs as the objects pass sequentially through the slit and fringe scan beams is collected by a microscope objective. The NBC scatter and fluorescence from the slit-scan region is focused through a slit aperture. The collected scatter from the fringe-scan beams is collected by the same objective but focused on a pinhole spatially separated from the slit scan aperture. After this pinhole, the fringe light is picked off by a ½ mirror and is then collimated by a lens and split by a dichroic filter onto two photomultiplier tubes. The slit-scan and fluorescence light is re-collimated and then split by increasing wavelength with a series of dichroic filters so that each scatter and fluorescence signal goes to the appropriate detector.

There are a number of technical challenges in the design, including complex illumination optics, the difficulty in alignment of combined laser illumination paths, and stability of the fringes over time. Flow conditions will be controlled accurately and precisely to minimize the core diameter and assure repeatable resolution performance. Furthermore, because high optical resolution combined with high flow rates is required, high detection bandwidth requiring new high speed amplifiers is necessary. The bandwidth requirements should not apply to fluorescence since these channels do not require micron resolution.

5.1.4 Nanobar Identification and Software Development

The identification of individual particles in flow requires the ability to reject particles that are out of focus, aggregated, or misaligned to the flow stream, since all of these could give incorrect identities. Aggregates are gated via a blob analysis and a “skeletonizing” algorithm (Callida Genomics, Sunnyvale, Calif.) where a blob is thinned to a line. Single particles result in straight and continuous lines. In flow data none of the information will be available in images to determine if a single particle is being viewed. Once fringe illumination is used the signature of a single particle will become even more complex. A slit scan channel will be used to get general particle shape information. The slit scan channel will be separate from a fluorescence slit channel. Both the scatter and fluorescence slit scan channel information will be key to sort out single events. Scattering signal is proportional to particle size. Aggregates may give a pulse length corresponding to a single particle but these will also produce a large scattering signal. So it is expected to be able to gate on signal strength. Forward scatter signal for reflecting particles is weak (Bohren et al., Absorption and Scattering of Light by Small Particles, Wiley, 1998, herein incorporated by reference). Aggregates will have larger forward scatter signals than single particles. Fluorescence signals will also be larger for aggregates. If the particles do not all completely align a wide distribution of pulse widths and heights may be seen. Again pulses based on pulse width and height will be selected.

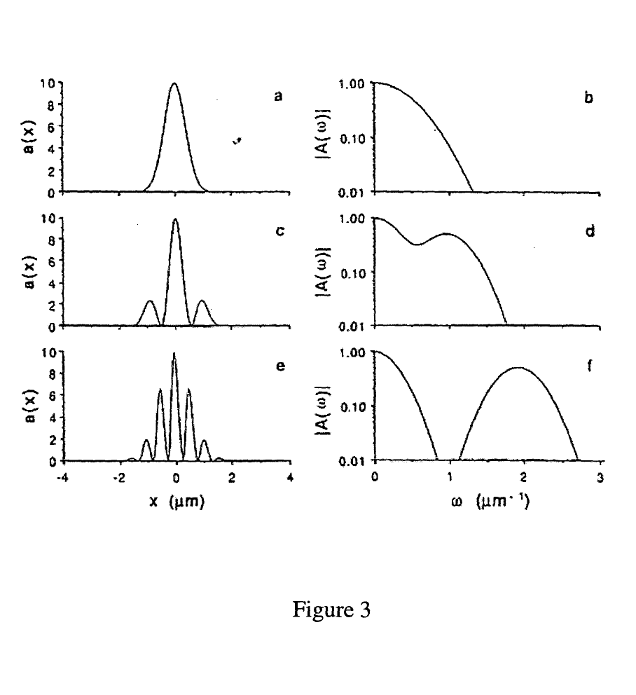

The fringe pattern increases the spatial frequency content of the imaging system and can produce nulls in the frequency response. FIG. 3a depicts a slit scan illumination profile and FIG. 3b shows the frequency response of this profile. FIG. 3e depicts a fringe profile and FIG. 3f shows the frequency response. The fringe profile contains a point where the frequency response goes to zero. The null point means that certain features would be missed by the system. Solutions to this problem include creating a chirped profile (a fringe pattern with multiple fringe periods) or using two fringe illumination schemes with two different periods. Since the slit response in FIG. 3b overlaps the fringe null in FIG. 3f, the latter is assessed using the slit and fringe channels.

SurroBar™ software (SurroMed, Mountain View, Calif.) is a nanobar identification program that accepts batches of reflectance and corresponding fluorescence images. The software searches images for particles, determines which particles are single rods, extracts line profiles of these rods, and compares them to a library of binary profiles. The comparison is a simple correlation that produces a coefficient from 0 to 1. The particle is given the identity corresponding to the largest correlation coefficient. Each identified particle in the reflectance image has a corresponding particle in the fluorescence image. The SurroBar™ software performs statistics on the fluorescent image particles to determine mean fluorescent levels for each identified particle. Particle identity, mean fluorescence and other attributes are written to a file in a list mode format for later analysis.

5.2 Sequencing by Hybridization

Sequencing by hybridization (SBH) relies on the ability of one piece of DNA to precisely recognize and hybridize with its complement (see U.S. Pat. No. 5,202,231 to Drmanac et al.; Drmanac, et al., Genomics 4:114 (1989); Drmanac and Drmanac, Methods Enzymol. 303:165 (1999), all herein incorporated by reference). In its simplest form, a complete set of oligonucleotide probes of a given length is exposed to a target DNA under optimal hybridization conditions. Those probes that hybridize to the DNA are identified and used to assemble the target sequences. Advantages of the SBH system include universal sequencing of any DNA sample, longer DNA read length, redundant data resulting from overlapping probes, and the opportunity for massive parallel processing in microarrays or solution arrays such as the NBC system (SurroMed, Mountain View, Calif.). The use of combinatorial probe sets, in which small sets of short probes are ligated (in the presence of matching target sites) to create exponentially larger sets of long probes has greatly increased the informational power of the test (Drmanac and Drmanac, DNA Array Methods and Protocols vol. 170 (2001), herein incorporated by reference).

One embodiment of the invention utilizes The HyChip™ system (Callida Genomics, Sunnyvale, Calif.) which consists of facing glass microscope slides containing four replica arrays of 1024 oligonucleotide probes consisting of all possible 5 base combinations. These bound 5-mer arrays are exposed to DNA samples and a complete set of 1024 TAMRA-labeled 5-mers, combined in various pre-mixed probe pools containing 16 to 256 probes per pool. The HyChip™ universal system can score a complete set of over one million 10-mer probes per sample using only 2000 5-mer probes.

5.3 Universal Selective Genome Amplification

Universal genotyping can be performed using universal adapters to amplify specific genomic regions to create pools of independent amplicons to be tested for the presence or absence of polymorphisms. Two, usually complete, sets of short universal probes (5-7 bases in length) and a ligation process are used to test any SNP. The most important element of this universal system is the ability to select any probe pair or any set of probe pairs for testing SNPs of interest. The use of NBCs allows for a higher capacity and flexibility. Hundreds of ligation assays can be handled per well in a 384-well plate format. Up to 150,000 wanted SNPs per individual human DNA sample can be tested in one plate. An automated plate handling and reading system has the capacity to test thousands of individuals for biomarker discovery and assembly of a knowledge base for the development of improved personal treatments and preventative medicine.

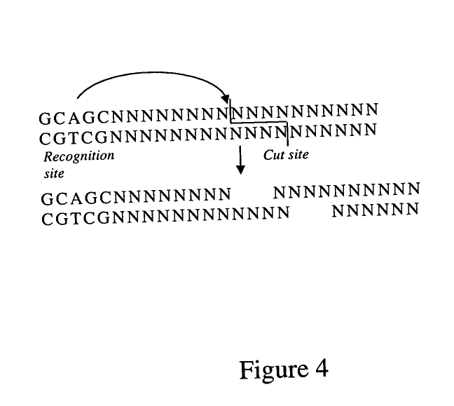

In the present invention, genomic DNA is cut with 2, 3, or 4 restriction enzymes having a 4-, 5-, or 6-base recognition site and that cut more than one base away from the recognition site to produce 4- or 5-base sticky ends (examples of such restriction enzymes include Hga I, Bbv I, and SfaN I) (see FIG. 4). This digest produces 10⁷ fragments of 100-300 bp in length. It is possible to cut the genomic DNA with a restriction enzyme that has a 5-base recognition sequence but leaves blunt ends in order to destroy longer fragments.

The present invention provides methods for isolating and amplifying specific 100-250 bp fragments of genomic DNA using Type US restriction endonuclease digestion of the genome to produce all possible overhangs of a given length. The frequency of each enzyme cutting at the five base recognition site is about once in every 1000 bp of sequence; however, because of the non-palindromic nature of the recognition sites for many of the enzymes, the opposite strand also contains recognition sequences, therefore the frequency of cutting will be about one cut in every 500 bases. Including a second or third Type IIS restriction endonuclease in the digestion mix will double or triple the frequency of cut sites and produce fragments of 250 to 125 bp in size, respectively. The sticky ends of the genomic fragments are ligated to adapter molecules, which can be either single-sided (a double-stranded piece of DNA, approximately 60 bp in length, that has one end which is complementary to the sticky end of the genomic fragment and the other end blunted or blocked) or double-sided (a double-stranded piece of DNA, approximately 60 bp in length, that has two ends which are complementary to the sticky ends of the genomic fragment). The adapter/fragment DNA is linear when using a single-sided adapter whereas it is circular when using a double-sided adapter.

The formation of closed DNA circles consisting of an adapter and a genomic fragment results from selecting specific sets of adapters that are complementary to both the overhangs generated within the genomic fragments (i.e. universal double-sided adapters) and thereby reduces the complexity of the ligation product. If one or two single-sided adapters are ligated out of two sets of 256, about 200 fragments will have a primer on each side, then a 50% success rate in the PCR amplification is required to obtain 100 amplicons/reaction. Alternatively, double sided adapters (one adapter with a sticky end on each end) can be ligated to the fragments resulting in the formation of a circular piece of DNA. The circularized or blocked DNA is selected for using exonuclease digestion followed by polymerase amplification. Fragments that either have an adapter on one side only or no adapters at all are eliminated by treating the samples with exonuclease. Exonuclease digests DNA from unprotected ends. Adapters that have modified ends or that result in circle formation protect the DNA from exonuclease digestion.

The adapter/fragment molecules are amplified using either Inverse PCR or rolling circle amplification (RCA). Inverse PCR allows the amplification of DNA segments that lie outside the boundaries of known sequence. PCR primers derived from the adapter sequences in inverse orientation are used such that the genomic sequence in the circularized molecule will also be amplified (Silver, “Inverse polymerase chain reaction” In, PCR: A practical approach, McPherson, et al., eds., Oxford University Press, New York, pp. 137-146, 1991, herein incorporated by reference). RCA uses either primers specific for the adapter sequences or random hexamers for the priming of the polymerase (Dean et al., Genome Res. 11: 1095-1099 (2001), herein incorporated by reference in its entirety).

A second round of digestion and adapter ligation can be done before or after PCR amplification and exonuclease digestion of unprotected fragments. If it is done before the PCR, more genomic DNA is required. One embodiment of the invention provides for ligation of 1-20 amplicons, whereas an alternative embodiment of the invention provides for ligation of multiple amplicon pools of 2000-20,000 in the first reaction which are subsequently subdivided into 10 to 100 or more fractions. In this case, two different primers are used per amplicon for single-strand production.

Adapter synthesis takes advantage of the combinatorial use of a limited set of oligomers to allow the complexity of the genome to be addressed using of unique sequences of 8 to 16 bases that specifically define individual genomic fragments. These single sequences are isolated without the need for custom primers. Coupled with universal genotyping and based on ligation of three probes selected from universal and complete libraries of 5-7-mer oligonucleotides, the system allows preparation and genotyping any single SNP or set of SNPs among the tens of millions that exist in any species. The present invention eliminates the enormous cost of making and handling millions of pairs of SNP-specific primers or probes.

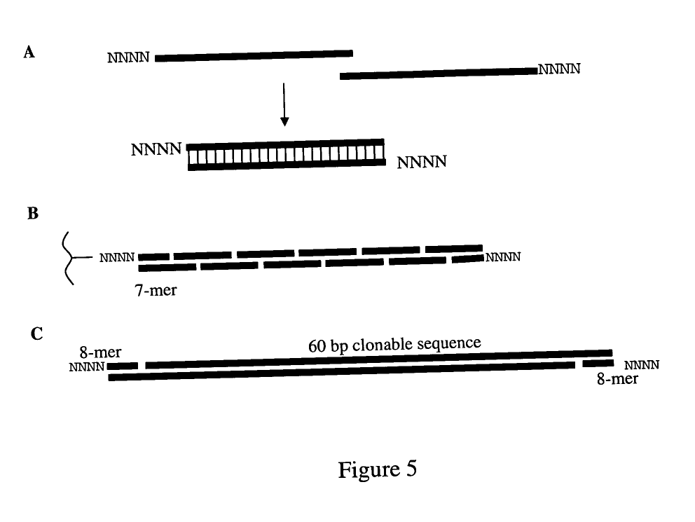

The nature of the overhangs for specific genomic fragments resulting from the enzymatic digestion described above can be predicted because the surrounding sequence of the polymorphisms of interest is known. A series of purification steps using adapters with complementary sticky-ends to the ends of the fragment is used to isolate the polymorphism of interest. A set of 256 single stranded oligonucleotides with sequences complementary to a second set of 256 oligonucleotides can be used to create all 32,768 possible combinations of double-stranded adapters (see FIG. 5). In one reaction vial, an adapter (or multiplicity of adapters) with specific four-base overhangs at each end ligates to fragments within the digested genomic DNA that possess complementary four-base overhangs.

An advantage of this system is that it is expandable to any number of SNPs (1 to 1×10⁷) because there is no need for specific SNP-specific primers. This system also allows for changing the SNP set and is a good method for discovering new SNPs because of the 100×100 base amplicon mixtures.

5.3.1 Adapter Construction

Several strategies can be used to construct the adapters. It is unnecessary to synthesize each half of the adapters as one complete oligomer and anneal them to each other, because each adapter need only differ by the four variable bases. In addition, intervening sequences that are common to every adapter do not need to be re-synthesized for each adapter. The preferred embodiment for double stranded adapter synthesis is to use selective annealing of a library of oligomers that are seven bases in length to an immobilized initiating support. In this way, specific elements of the adapters, such as primer binding sites or restriction enzyme sites, can be introduced and swapped out as needed. In an alternative embodiment, an initial 60 bp of clonable sequence is synthesized and cloned into a vector. This common core to the adapters contains a restriction site at each end so that after release from the vector and purification, a library of 256 8-mers can be annealed and ligated onto the core sequence. In another embodiment, adapters are synthesized with degenerate oligonucleotides resulting in one variable base within the 4-mer sequence incorporated during synthesis of the oligonucleotide instead of four fixed bases at each end of the adapter with the 32,000 variants. Therefore, there will be 4^{6÷2} variants or 2048 oligonucleotides resulting from only 2×64 synthesis reactions. Starting with 16,000,000 fragments, the first round distributes the fragments into fractions with 512 adapter types. The second and third rounds apply selection to the SNP fragments by adding in adapters for the SNPs present, e.g. 250 selected adapters from a possible 512 adapters result in a 2-fold enrichment.

Two sets of primary oligonucleotides are needed. Each set has at the 5′ or the 3′ end all possible 3-mers, 4-mers, 5-mers, 6-mers, or 7-mers and a connecting segment of approximately 8 to 20 bases. The connecting segment can be different for each set or even for subsets within the set. Two to four sets of 4 to 64 internal connectors may be used, each with complementary matching sequence to the connecting segments in the primary oligonucleotides. These segments allow making different connections between primary oligonucleotides and may allow the formation of 10 to 100 specific double adapters in one mix. They can have either restriction sites, for ligation and the second round of digestion, or primer sites for PCR amplification. The central connectors are made to contain the proper restriction sites of primer sites.

5.3.2 Ligation of Adapter to Target DNA

In a preferred embodiment of the invention, at least two restriction enzymes are selected that recognize 5 to 7 bases and generate a 5-base overhang (sticky end) to digest the genomic DNA and be complementary to the sticky ends of the adapters. The adapters are added to the genomic fragment (two single-sided universal adapters to make a linear molecule, or one double-sided universal adapter to make a circular molecule). In the first reaction, the genomic DNA is digested, ligated to the adapters (the addition of carrier DNA may be necessary) and treated with exonuclease. Alternatively, the adapter can contain a 6 to 7 base recognition site to open a more specific sequence of 5 bases if unwanted fragments are being amplified. The product is amplified by inverse PCR or RCA and then undergoes a second round of digestion, ligation to selected adapters, and amplification.

Alternatively, 4-base sticky ends can be used in two reactions. Starting with 1×10⁶ fragments, the addition of 5 to 10 pairs of adapters gives 500 to 2000 protected fragments. Digesting with a second set of restriction enzymes from each end and ligating with 10 additional pairs of adapters results in 0.25 to 4 unwanted amplifications. Again, a two-step process is used. In the first step, the genomic fragment is ligated to an adapter containing a restriction enzyme recognition site for a different set of restriction enzymes than those used in the original digestion, followed by exonuclease treatment. In the second step, the amplicon is digested with the second set of restriction enzymes and ligated to a universal adapter containing primer sites and is amplified. Using two sets of 256 universal adapters (those that contain restriction sites or primer sites) the first and second steps can be carried out in the same tube; however, it may be necessary to use overhangs of different lengths.

The method of the invention uses a restriction enzyme site to linearize the adapter prior to amplification with Taq or Vent DNA polymerases. Another embodiment of the invention incorporates uracil into the sequence. Subsequent treatment with uracil-DNA glycosylase and heating will break the DNA at the uracil insertion point thereby achieving the same goal as restriction enzyme digest to enhance amplification.

In a genomic fragmentation mixture of 16,000,000 pieces of DNA that have been ligated with an adapter with two four-base overhangs, approximately 488 unique fragments are predicted to be captured onto both ends of the adapter (16,000,000 fragments divided by the number of possible combinations (32,768) of a specific 4-mer at one end of the fragment and a specific 4-mer at the other end, regardless of orientation).

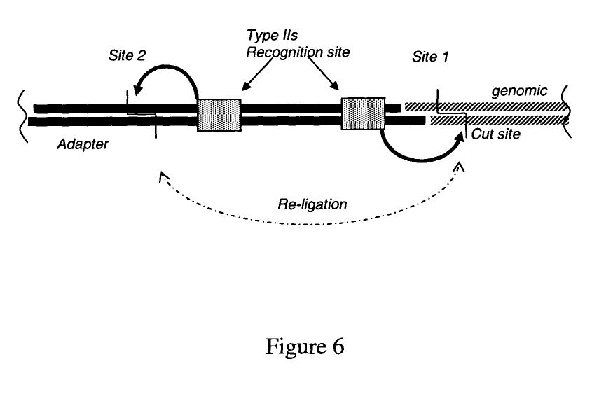

In a modification of the preferred embodiment, the adapter is prepared such that two Type IIs restriction sites are incorporated into the adapter. One site is positioned to cut into the adjacent genomic sequence to form an overhang sequence generated from the genomic sequence (“site 1 overhang”). The second site (“site 2 overhang”) is positioned such that it cuts into the adapter to form an overhang sequence that is complementary to the genomic sequence generated by the first site (see FIG. 6). Ligation of one end of the molecule to the other will therefore reform a circular molecule. For a 4-base overhang selection, 512 adapter oligonucleotides are prepared and a pair is selected to generate an adapter (one of 256 variants). The adapter contains the 4-base sequence that is complementary to the overhang formed at the genomic cut site.

Primer sequences are located in the adapter outside of the region that will be lost by digestion and re-ligation. The same adapter can therefore be fused to provide an increased level of selection by cutting and re-ligating without incorporation of a new adapter.

With the internal-cut adapter strategy of digesting genomic DNA with a first restriction enzyme followed by cutting with a second restriction enzyme, it is possible that if there are sites within the genomic insert that are recognized by the second enzyme, there is a 50% chance that the SNP is contained within the fragment that is not captured and amplified. To overcome this possibility, the method of the invention provides for using the same restriction enzyme to cut the genomic DNA as well as for the second cut after capture. However, there is a 50% chance that by using the same restriction enzyme the original site is reopened because the recognition sequence may have been located on the captured fragment. To overcome this possibility, the method of the invention provides for using a methylase enzyme to block sites in the genomic DNA after the first genomic digestion. Only the recognition sites within the adapter would then be recognized when the second cut is made with the same enzyme as the genomic digest.

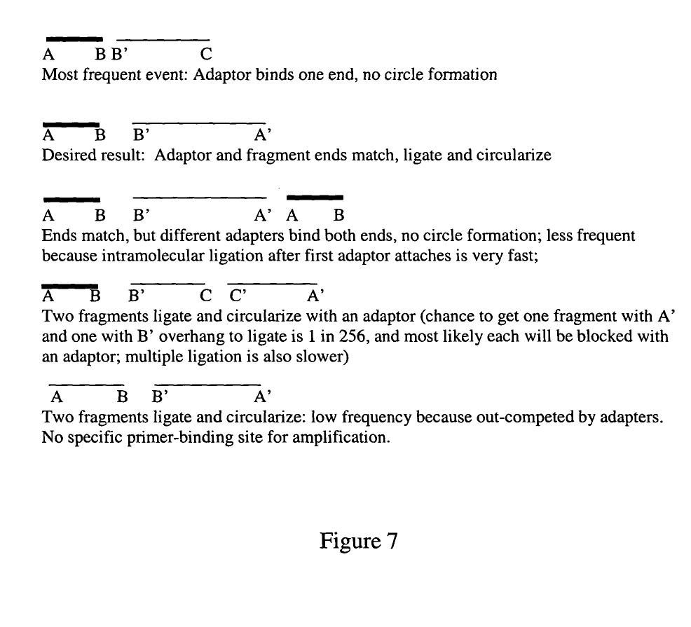

With any specific adapter, a variety of possible ligation events can occur (see FIG. 7). The desired event is formation of an adapter-genomic fragment circle, in which the two ends of a genomic DNA fragment match the two ends of the chosen adapter. The formation of closed circles of DNA allows for several possible approaches to the selection of the desired fragment from unwanted fragments. Exonuclease treatment requires exposed 5-prime or 3-prime ends or breaks depending upon the enzyme chosen before the DNA will be degraded. Linear, non-circularized DNA can therefore be removed by exonuclease treatment of the sample.

In another embodiment of the method of the invention, biotinylated blocking adapters are used rather than exonuclease to remove non-circularized fragments. Biotinylated blocking adapters, which consist of all possible combinations of overhangs, are included in the ligation mix. For a 4-base overhang, a mixture of 256 blocking adapters is used that have equal representation of each specific 4-base sequence. The other end of the adapter is not able to ligate and is biotinylated, which provides a mechanism for removal of bound fragments through binding to streptavidin-coated beads that can be collected by magnetic attraction or centrifugation. The concentration of blocking adapters is in excess to that of complementary genomic ends to ensure removal of free adapters, unligated genomic fragments, and ligated adapters/genomic fragments that have not formed circles. Additionally, the digestion of the first adapter and ligation of the second adapter could occur simultaneously if the second adapter does not contain restriction sites found in the first adapter.

Rolling circle amplification can be used to specifically amplify the circularized DNA. The amplification can be primed with either random hexamers or specific primers, and in this scenario, primers could be designed to a region within the adapter. Another possibility for amplification is inverse PCR in which the forward and reverse primer binding sites are located in the adapter, but both primers are extended out from the adapter in opposite directions resulting in the formation of amplified linear DNA from the fragment sequence.

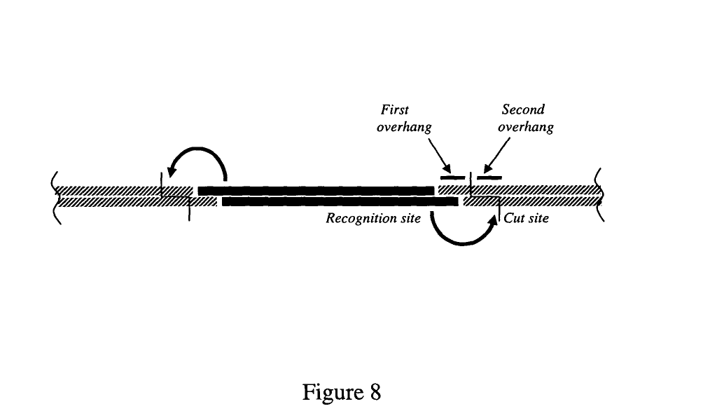

The method of the invention provides for a second step of opening the circles generated in step one and ligating new adapters to generate smaller pools of fragments, or to isolate and amplify a single DNA fragment of interest. To achieve this enhanced sequence specificity of genomic fragments each primary adapter contains two recognition sites for a Type IIS restriction enzyme to result in a new cut site(s) within the genomic DNA (See FIG. 8). The principle is that a second cut within the genomic sequence, coupled with an appropriate secondary adapter, creates a new opportunity to achieve sequence specificity.

In the preferred embodiment, 144,000 SNPs will be selected from a set of 600,000 specific high quality predefined SNPs and have them in a form that allows genotyping with NBCs within one 384-well plate. To achieve this requires minimizing the sequence complexity (removing non-SNP containing fragments) of the fragments to reduce the possibility of false positives to an acceptable level. It is likely the 144,000 SNPs will fall within fragments of all possible 4-base overhangs so the initial round of selection will use a pool of approximately 400 adapters selected from all possible 32,000 adapters. The adapters in each well are specifically selected for SNPs that possess a particular 5-base sequence flanking the SNP. These five bases will be used later in the genotyping phase so that a common labeled probe can be used in each well. However, it is likely that each well will have several labeled probes per reaction well so that each SNP can be interrogated by more than one attached probe, or that SNPs not sharing the same 5-base labeled probe sequence can be examined in the one reaction well.

Another embodiment of the invention digests the genome with a blunt-end restriction enzyme to remove about 75% of fragments before the first round of selection by producing blunt-ends in many of the longer fragments. With 360 reaction wells, 24 control wells and 400 specific adapters added to each well, it is predicted that 48,800 fragments per well would be captured. The specific SNP fragments are further purified by the adapter-initiated second cut into the genomic sequence and re-ligation to a new adapter that results in about 610 unwanted fragments per well, but maintains the selected 400 SNP fragments. The total sequence complexity is now about 100,000 bp with 400 SNPs per well. This low degree of sequence complexity coupled with the use of specific labeled probes will enable the accurate and rapid scorning of at least 144,000 SNPs in a single 384-well plate.

The use of double-sided adapters can give rise to false positives. If 100 adapters are used containing different 5-mer sticky ends, then the number of false positives from 16,000,000 fragments will be 16×2×100. However, the second step greatly reduces the number of false positives to 1600/10,000 or ⅓. If there are 1,600,000 fragments for one amplicon there may be only one additional false positive.

To eliminate non-specific ligation events, the post-ligation reaction is treated with an endonuclease that recognizes mismatches in the DNA, thus destroying the structure. Alternatively, a Type IIS restriction enzyme cleaves a recognition site in the adapter to reproduce the original overhang and the ligation event is repeated followed by exonuclease treatment resulting in successive rounds of purification. By repeating the ligation with the same adapter sticky ends, the removal of fragments with mismatched sticky ends is improved exponentially. A 100-fold purification in one step will give 10,000-fold in two steps and 1,000,000-fold in three cycles. This multi-cycle ligation step may also remove circles with multiple inserts or multiple adapters. Repeated cycles of cutting and ligation require clean and efficient enzymes to prevent sticky end degradation and assure more than 70% success in circularization of matching fragments.

It is possible that the ligation can occur on one side of a fragment containing two breaks due to a mismatch on the other side. In this case, endonuclease enzymes that recognize breaks in the DNA can be used to remove this fragment. One way to avoid this problem is to use restriction enzymes with different 3′ and 5′ sticky ends so that most of the fragments will have a different primer. It is possible to make multiple adapters for the same 4-mer sticky end to allow for primer mixing and may facilitate amplification. To further protect against exonuclease degradation, adapters with protective groups can be used.

Two secondary cuts are made to release the adapter. This should not interfere with the ligation because the ratio of released adapter to secondary adapter is low. In addition, the secondary adapters are prepared with appropriate priming sites that do not allow cross reactivity with the primary adapters in the amplification reactions. To avoid uneven amplification due to fragment size and sequence differences starting with more than the necessary number of SNPs so that the number of unsuccessful SNP fragments is tolerable.

In an alternative embodiment, all possible combinations of 4-mers on each end of the adapter may not be included due to the likelihood of undesirable ligation events, such as when the 4-mers at each end of the adapter are complementary to each other. Because each 4-mer on the adapter cannot have its complementary 4-mer on the same adapter, 255 possible combinations result for each 4-mer (256-1=255). Elimination of all complements with single base mismatches in the complement, but not at the ligation base, leaves 246 (256−1−9=246) possible combinations for a single 4-mer. Adapters with palindromic 4-mer overhangs may ligate inter-molecularly (e.g. AATT would ligate to the same sequence on another adapter) and become unavailable for ligation to genomic fragments. There are 16 possible 4-mers that fall in this category.

The invention provides a method to avoid adapter self ligation by using adapters that are not phosphorylated or that incorporate a site that destroys the linkage if it should form. The use of non-phosphorylated adapters prevents the formation of adapter-adapter linkages. Adapter-genomic linkages may form but contain a nick in one of the strands that will be repaired by base replacement. Alternatively, it may be useful to perform genomic fragmentation with different groups of enzymes so specific polymorphisms will be located on different fragment types that can be selected with alternate adapters that do not self-ligate.

In another embodiment, a universal set of adapters with one blocked end, such as a phosphorothiolate bond that is resistant to exonuclease digestion, is used. In this case two adapters have to be ligated on each side of the matching genomic fragment and fragments of interest will be linear but protected from degradation by an exonuclease.

5.4 Universal Genotyping with Combinatorial Probe Sets

The ability to identify over 100,000 SNPs in a single plate is a dramatic improvement in SNP scoring over competing technologies, resulting in substantial cost savings and improvements in multiplexing capability. In principle, all (currently known or yet to be discovered) medically important SNPs in a patient sample, not just 100,000 can be identified. The same libraries of probes are applicable to all other species besides humans, including those that are important for agricultural biotechnology and other industries.

To achieve the sequence specificity necessary to identify unique sites within the genomic DNA, the preferred embodiment of the present invention utilizes a three probe ligation strategy featuring complete libraries of fixed hexamers, 7-mer internal spacer probes, and 5-mer labeled probes. When assembled, these three probe types create unique 18-mer labeled probes, reducing the chances of two positive sequences occurring within a single well to virtually zero.

Promising results have already been achieved using universal probe sets on encoded microparticles such as the Luminex 100 system (Luminex Corp., Austin, Tex.). Expanding these results to 1000 or 4000 NBCs will represent a significant improvement, greatly increasing the multiplexing capability of the universal genotyping system. By utilizing hexamer probes on 1024 to 4096 different NBCs and up to 400 SNPs in a single well or up to 153,600 SNPs in a single 384-well plate can be identified.

For 1000 to 4000 NBCs, the number of probes to use is as follows: 4000 individual 6-mers and 16,000 individual 7-mers which are bound to the NBCs (one NBC is used four times), and 4000 labeled 6-mer probes, 2000 of each color.

The method of the invention utilizes probes based on the following design: fixed probes 5′-NH₂-Spacer-Spacer-NNN123456-3′, internal probes 5′-pl234567-3′, and labeled probes 5′-p12345NN-Fluorophore-3′, wherein the numbers represent sites of specific informational bases and N represents equal molar ratios of all four bases. Certain probes within a complete set of probes may vary for the standard design to help equalized hybridization/ligation potentials. All probes will be tested via SBH-based assays on HyChips™ (Callida Genomics, Sunnyvale, Calif.) to assure optimization.

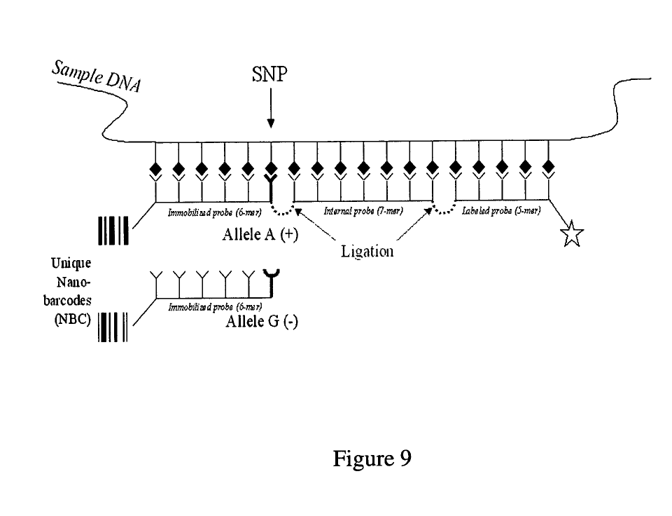

One embodiment of the invention is to use combinatorial ligation of three or more probe sets that may be represented by pools of probes to score large numbers of complete sets of probes longer than 10, 11, 12, 13, 14, 15, 16, 17, 18, 19, 20, 21, 22, 23, or 24 bases (see FIG. 9). The first set of probes (or probe pools) may be attached to a support, and the last set of probes (or probe pools) is labeled. Internal probes (or probe sets) may be labeled or unlabeled and if they are labeled, they may represent donor or acceptor molecules for FRET analysis of the probes in the ligation construct. The configuration is as follows:
(support)−(spacer 1-4)−5′N0-3B5-8−(ligation)−B5-7−(ligation)−B2-6N03—label

The first set of probes is fixed, meaning that they are bound to a support, such as a glass plate or bead, by a spacer. The second set of probes is free, meaning that they are in solution and are used as a linker between the fixed and labeled probes. The free probe has to be long enough to allow two ligations, as the ligation molecules are larger than the size of one nucleotide. The third set of probes is labeled, meaning that the probe is conjugated to a fluorophore, such as fluorescein, and is also in solution. The labeled probes can be combined into one pool. The second ligation would then serve to improve the discrimination of the 3′ end of the free probe. In this configuration, chaining of the free probes gives the same positive signals as the unchained probes (1/M=1, since M=1).

In the preferred embodiment, the fixed probe is attached to an NBC and each support can hold more than one probe; therefore, a complete set of fixed probes is obtained. The complete set of labeled probes is separated into N (wherein N=4 or more) informative pools. The complete set of labeled probes is separated into M (wherein M=4 or more) informative pools. The number of label colors is C (wherein C=1 to 4¹⁰). The total number of possible combinations of free and labeled pools is N×M. The number of required replicate arrays is R=N×M/C.

A sample is added to each of the N×M different combined pools. Each of the combined pools plus target is applied to N×M arrays. The free probe will hybridize to the target and ligate to the free probe. The labeled probe can also hybridize and ligate to the fixed probe, but since it is shorter than the free probe, it will give a smaller signal. Shorter probes are less stable than longer probes. It is also possible to have multiple free probes that ligate together in a chain in front of the labeled probe, resulting in a false positive. The frequency of one extra free probe signal is 1/N, the frequency of two extra free probes is 1/N², and so on for more free probes. There are 1/M chances that the labeled probe after a chain is in the same pool as the labeled probe would be after only one free probe.

One benefit of two ligations is improved discrimination of the bases near the ligation site. Reduced discrimination far from the ligation site limits the informative length of the labeled probe to 5 or 6 bases. This is partially why an N-mer of informative length (greater than or equal to 12 oligonucleotides) with only one ligation is not usable. The other reason is that it is expensive to handle a complete library of all 8-mers (or larger N-mers). With two ligations, each of the probe libraries can contain shorter probes.

Probe set production is evaluated for yield by optical density, purity by capillary electrophoresis, and presence of expected mass of full-length products by matrix-assisted laser desorption/ionization (MA/DI) mass spectroscopy. Oligonucleotide probes will be subjected to Callida's semi-automated robotic matriculation process, which includes: database input, master replica aliquoting, SBH quality control (QC), and −80° C. cold storage.

The HyChip™ (Callida Genomics) will be used as a high throughput analysis system to QC probe libraries based on the SBH process. Specifically designed synthetic DNA target libraries will be acquired to test each SBH probe using informational ligation events. Callida QC includes evaluation of all probes for concentration by optical density and spectral shape, nuclease activity by incubation and subsequent gel analysis, and sequence verification via SBH. Fixed probes will also be tested for relative attachment efficiency to HyChip™ using mass hybridization analysis. Labeled probes will be evaluated for active fluorophore incorporation and spectral analysis, as well as compatibility in a SBH hybridization/ligation enzymatic assay. The high throughput nature of HyChip™ technology allows multiple replicate (typically eight) experiments for each probe. Full-match and mismatch signals for each probe will be determined using the universal set of targets.

Combinatorial ligation of short probes to generate longer informational probes is a fundamental advantage of Callida's universal genotyping technology. The preferred embodiment of the invention incorporates a full set of barcode-attached hexamers, combined with full sets of 7-mer spacers and 5-mer labeled probes. This generates unique 18-mers, which occur so infrequently in the genome that it is statistically expected that no false signals will occur in a given reaction. Such combinatorial probe design minimizes the need for multi-step sample preparations aimed at reducing sample complexity. The results of these experimental tests will define sample preparation requirements.

The preferred 3-piece ligation strategy will use both the HyChip™ and NBCs using synthetic targets and genomic amplicons. A limiting amount of target DNA ˜1-100 amol), 20 replicate fixed probe (four for each SNP) and a 20-fold excess of internal probe are combined under SBH hybridization/ligation conditions followed by addition of 10-fold excess of labeled probe. Alternatively, all probes are combined and the reaction is controlled through thermal manipulation of kinetics. After the SBH reaction is complete, the NBCs will be analyzed by the SurroMed detection system for barcode identity and corresponding signal intensity. The raw data output will be subjected to Callida's proprietary algorithms to understand informational ligation events.

Single and multiple samples will be tested on the HyChip™ to determine the effects of multiplexing samples, probe pooling, and reagent concentrations. Previous experience with micro-particle SNP analysis suggests that probe and sample concentrations, temperature, enzyme, and sequence interactions can affect signal-to-noise ratios and specificity, specifically variation of solution phase probe and target concentrations. These studies also indicate that data obtained from encoded micro-particles correlate well with or even exceed data quality obtained from the HyChip™. Results from these studies on NBCs will be used to optimize final reaction conditions and system capacity.

5.4.1 Fixed Probe Attachment

The present invention provides methods to attach aminylated oligonucleotide probes to carboxylated NBCs through carbodiimide catalyzed amide bond formation. Typically, a ten-fold excess of probe to carboxylated sites is required, along with sufficient concentration (>1 μM) to achieve high yield. Attachment efficiency is determined through Callida's mass hybridization process, which reacts with all probes with nearly equal efficiency. Many different probes can be pooled and tested simultaneously. The pooling process equalizes signal strength between different probes by eliminating variations in the reaction buffer conditions. Important factors to consider are the homogeneity of intra-and inter-barcode attachment, dynamic signal range, stability of attachment and barcode recovery. It is expected that variations up to 20% can be corrected by normalization factors provided by mass hybridization reactions. Previous experiments with micro-particles has demonstrated robust chemical condensation of amides with free carboxyl groups, but also indicate reduced recovery of particles after several wash cycles. SurroMed high-density particles will facilitate recovery through gravity precipitation. Once individual reaction conditions are optimized, the process will be semi-automated to allow high throughput attachment.

The method of the invention provides for all amino-modified oligonucleotide probes from a complete set of N-mers to be attached to specific NBCs and arrayed in multi-well plates. If the number of probes is greater than the number of available barcodes, the barcodes or probes may be pooled and assayed in separate wells. Each reaction is subjected to QC analysis (via mass hybridization) and probes with less than an 80% expected signal are re-attached. Individual reactions are aliquoted to allow analysis of individual or multiplexed barcodes. Alternatively, multiple reactions are analyzed using a multiplicity of barcodes compatible with robotic handling to allow high throughput analysis. Even though it is expected that there will be only 4096 unique fixed probes attached to barcodes and they will be readily evaluated individually, this QC test also acts as a test of the multiplexing capacity of the system. Small variations in attachment efficiency between different NBCs (up to 20%) will be compensated for by normalization, a necessary process for accurate data analysis.

Upon optimization of the signal-to-noise ratio for single SNPs, the sample complexity will be increased to meet multiplexing requirements for the study and the reaction process will be streamlined to reduce handling and reaction times. The robust nature of SBH suggests that optimized HyChip™ conditions provide an excellent starting point for all experiments using NBCs. The number of different fixed probes in any one reaction has little effect of reaction results; however, the number of solution phase probes can significantly affect the rate of false positives. Thus, a strategy that tests several SNPs using the same labeling probe will be incorporated resulting in the elimination of false positives due to the large number of labeled probes. The same strategy can be applied to internal probes or to limit both internal and labeled probes. Table 3 summarizes the reaction components and their specifications.

TABLE 3
Specifications for Genotyping Reaction

| | Range | | |
|---|---|---|---|
| Variable | Minimum | Maximum | Comment |
| General | General | General | General |
| Volume in uls | 1 ul | 100 ul | Plate may limit volume |
| SBH frames | 1 | 4 | Linear effect on all probe requirements |
| # of probes for each frame | 2 | 4 | Single base sequencing with 4 |
| Ligase | 0.001 unit | 1 unit | Effects kinetics and specificity |
| Sample | Sample | Sample | Sample |
| Moles of Sample | 10 fmol | 1 pmol | Based on previous studies |
| Individual Sample Conc. | 10 fmol/100 ul = 100 pM | 1 pmol/10 ul = 100 nM | Note: range of ranges |
| # Samples Multiplexed | 10 | 400 | Also affects sample preparation requirements |
| Pooled Sample Conc. | 1 pM | 100 nM | Note: expansion of ranges |
| Fixed Probes/Barcodes | FP/Barcodes | FP/Barcodes | FP/Barcodes |
| Average # of replicate Microparticles | 10 | 100 | Based on statistical requirements |
| Sites per Microparticle | 10,000 | 100,000 | Dependent on surface area |
| Moles of each Fixed Probe | 0.016 amol | 0.16 amol | Expect to use 10 fold excess to attach |
| Conc. of Fixed Probes | 0.01 amol/100 ul or 0.1 fM | 0.1 amol/10 ul or 100 fM | Not homogeneous |
| Surface Conc. of Fixed Probes | >1.0 uM | <1.0 mM | Conc. dependent on size of solvation sphere |
| Labeled Probes | Labeled Probes | Labeled Probes | Labeled Probes |
| # Labeled Probes per reaction | 4 | 40 | Minimize by pooling proper |
| | | | samples |
| Types of fluorophores | 1 | 4 | May not be necessary |
| Moles each Labeled Probe | 10 fmol | 1 pmol | Cost limited |
| Conc. of each Labeled probe | 100 fmol/100 ul or 1.0 nM | 1 pmol/10 ul or 100 nM | Can affect kinetics dramatically |
| Conc. of Pooled Labeled Probes | 4 nM | 4 uM | Can affect kinetics and specificity |
| Internal Probe | Internal Probe | Internal Probe | Internal Probe |
| Moles of each Internal Probe | 100 fmol | 10 pmol | Based on labeled probe requirements |
| Conc. of individual Internal Probes | 100 fmol/100 ul or 1.0 nM | 10 pmol/10 ul or 1.0 uM | Can affect kinetics and specificity |
| # of Pooled Internal Probes | 10 | 400 | Based on sample Complexity |
| Conc. of Pooled Internal Probes | 10 nM | 400 uM | Can affect kinetics and specificity |

Once reaction specifications are determined, the process will be set up robotically in multi-well plates and analyzed in the same plate. For this task up to 1000 SNPs will be tested to demonstrate the accuracy expectation and reproducibility of the system and eliminate wash steps and any downstream handling. Once confocal imaging is achieved, high-density barcodes can be imaged at the bottom of the wells and the majority of background interference from unligated label will be out of the focal plane. Furthermore, dilution of the labeling reaction may reduce or eliminate washes. The labeled probe concentration will be greatly increased on the barcode due to ligation and can eliminate concerns of background signals completely.

5.4.2 High Throughput Ligation Genotyping

The preferred embodiment of the invention provides for specific amplification of 400 SNPs in a single well. Each SNP assay requires using at least four specific NBCs (full-match and mismatch for the SNP frame as well as full-match and mismatch for the adjacent frame), two internal probes and two labeled probes for each strand. Samples can be amplified that share the same internal or labeling probes thus reducing false negatives due to complexity issues. Furthermore, samples can be split for analysis of both strands. Studies will utilize up to 1000 genomic amplicons. Initially, the same sample can be tested in each well of a multi-well plate to evaluate maximum throughput of the analysis instrumentation and software processing of data. The next series of experiments will make use of different SNPs in each well of a multi-well plate to evaluate the system specification as well as SBH compatibility with the system. Wild-type and variant samples can be mixed and matched in a blind experiment. Samples will be pooled as multiplex samples and tested to evaluate the effect of multiplexing on signal strength and accuracy by comparing results of multiplexed samples to individual samples. Once the instrument analysis has been demonstrated with an individual plate and the SBH process is verified, the entire set up and analysis process will be robotically automated.

The method of the invention provides for a system for testing 10,000 predetermined SNPs. DNA targets appropriate for the test will be selected and samples will be prepared by the adapter methods outlined above. Genomic samples that will be used for these tests can be selected to have 10,000 SNPs confirmed by other methods. Alternatively, the results can be verified on at least a statistically significant subset of SNPs after the test is performed. Based on the empirically determined multiplexing capacity, the number of samples per well can be defined and the required number of wells to test ˜10,000 samples can be predicted. Barcodes with needed attached 6-mer probes and unattached internal and labeled probes will be selected from complete libraries that match the targets of interest and appropriate probes will be pooled for reaction set-up. A batch of prototype plates will be pre-aliquoted with the required amount of appropriate probes. Reactions will be initiated by adding sample and enzyme cocktail. Data obtained from these tests will be used to optimize specifications needed for identification of 100,000 SNPs from complex samples.

The method of the invention provides a system for designing and testing 144,000 predetermined SNPs. Target candidates will be identified and DNA samples prepared from at least two individuals. A batch of pre-aliquoted probes in 384-well plates will be used to create prototype kits. The samples will be combined with the appropriate probes and reaction cocktail. The final experiments will be conducted in multiple replicates (at least three) in order to verify reproducibility. The results will be carefully analyzed to determine success rate and document factors that can be further optimized in the commercial product development.

The use of unlabeled 6- or 7-mer probes as a middle probe is expected to be very efficient. However, it is possible that 6- or 7-mer probes will not be specific enough or that the labeled probe may hybridize and ligate directly to the NBC-bound 6-mer probe. Proper high temperature in addition to ligase-provided specificity for 2 to 3 bases at the ligation sites should prevent most of the mismatches, but may allow some ligation of the 7-mers with mismatches at positions 5, 6, and 7. This may even be favorable because all of the bound 6-mers will get full-match or mismatch or mismatch 7-mers ligated after enough time. Incorrect 7-mers, especially with mismatches at positions 6 and 7 that are now at the new ligation site will block ligation of labeled probes, thus preventing ligation of labeled probes directly to immobilized probes. Standard two-probe ligation can be used on 100 specifically selected short amplicons per reaction that share a few labeled probes. Alternatively, the middle probe can be labeled to apply energy transference in detection. If the 3′-labeled probe is ligated directly to the immobilized 6-mer it will prevent ligation of the 7-mer and it will not generate light of the proper wavelength without a nearby donor dye.

Some amplicons of 100-200 bases each (e.g. 400) may create a high background because of many mismatch sites or frequent occurrences of a full-match target for the same combination of immobilized and labeled probes outside of the polymorphism site. This problem is square dependent on a number of SNPs per reaction because total number of bases is proportionally higher (getting up to 100 kb) and a proportionally larger number of different labeled probes have to be used. Smaller amplification reactions or short amplicons that share a few labeled probes can be used. For example, 16 groups of 25 SNPs each with the same labeled 5-mer may be selected for one reaction thus reducing the number of required 5-mers and false signals 25-fold. 6-mers are very important because they have 1 to 2 instead of 2 to 3 N and should therefore produce four times stronger signal. Selection of labeling dyes that have high efficiency may be critical. Biotinylated labeling probes may be used and the signal from streptavidin conjugates of phycoerythrin may be scored. If more signal is needed, a dendrimer labeling schema with 16 or more dye molecules per probe may be developed. Quantum dots or nano-beads can also be used to increase sensitivity.

### 6. EXAMPLES

### Example 1

### Isolation of Fragments from E. coli

The isolation of specific fragments from a complex mixture of fragments was tested on the 4.5 Mb E. coli genome which, when digested with Bbv I, produces an estimated 18,000 fragments with variable 4-base, 5-prime overhangs. Three fragments were selected of 100, 150, and 200 bp in size from three random regions of the published E. coli MG1655 genome and adapters were designed and prepared for ligation with the digested genomic DNA (see Table 4).

TABLE 4

| Primer Name | Primer Sequence |
|---|---|
| 100 Left | GGTCGCTGCCATCCCCAA |
| 100 Right | TCAAGTCCCCATCCGCTGTCT |
| 150 Left | GTTGGCTGCCATCCCCAA |
| 150 Right | TTTTGTCCCCATCCGCTGTCT |
| 200 Left | TGTAGCTGCCATCCCCAA |
| 200 Right | CACCGTCCCCATCCGCTGTCT |

Adapters were prepared by annealing two complementary oligonucleotides that, when double stranded, produced 14- and 17-base, 3-prime overhangs. Two shorter, variable oligonucleotides were then ligated to the phosphorylated core with T4 DNA ligase to produce the 4-base, 5-prime overhangs. The complete adapter was phosphorylated and purified using the QIAquick spin protocol (QIAGEN, Germany) and the DNA was collected in 40 μl of Tris/EDTA solution. Adapter A contained the sequence for the following primers: 5′-TGAGACCACAGCCTAGACAGC and 5′-CTGCAAGGCGATTAAGTTGG. Adapter B contained the sequence for the following primers:

5′-GACGGCTGAAATTGGTAAGG
and
5′-CGGAATCAAAGCAGGATAAGG.

Adapter A (20 to 200 fmol) was ligated to 1200 ng of Bbv I digested genomic DNA in a volume of 10 μl for 30 min at 25° C. in the presence of 1×T4 DNA ligase buffer (New England Biolabs, Beverly, Mass.) and 200 U of T4 DNA ligase (New England Biolabs). The enzyme was heat-inactivated at 65° C. for 10 min. the ligation reaction (10 μl) was then treated with 1U of Bal-31 nuclease (New England Biolabs (NEB)) in the presence of Nuclease buffer for 30 min at 30° C. and then heat-inactivated.

PCR conditions for Adapter A were as follows: 94° C. for 3 min, denature at 94° C. for 20 min, anneal at 66-62° C. for 30 sec in the first 5 cycles and 62° C. for 30 sec in the following 35 cycles, extension at 72° C. for 30 sec. For the second step selection, Type IIs restriction enzyme digestion of the adapter was performed in a 10 μl reaction with 8 μL of purified PCR reaction, 1×NEB Buffer 2, and 1 μl (4U) Fok I enzyme. Digestion was for 60 min at 37° C. followed by inactivation at 65° C. for 20 min. The digest (8 μl) was then combined with 1 μl of T4 DNA ligase buffer, 1 μl Adapter B (20-200 fmol) and 200U of T4 DNA ligase. After incubation for 30 min at 25° C. and inactivation at 65° C. for 10 min, the reaction was digested with Bal-31 nuclease. The sample was then diluted 10-fold with Tris/EDTA (10 mM/01.1 mM) and 1 μl was used in a 30 μl PCR reaction at 94° C. for 3 min, 94° C. for 20 sec, 58° C. for 30 sec, 72° C. for 30 sec, over 35 cycles.

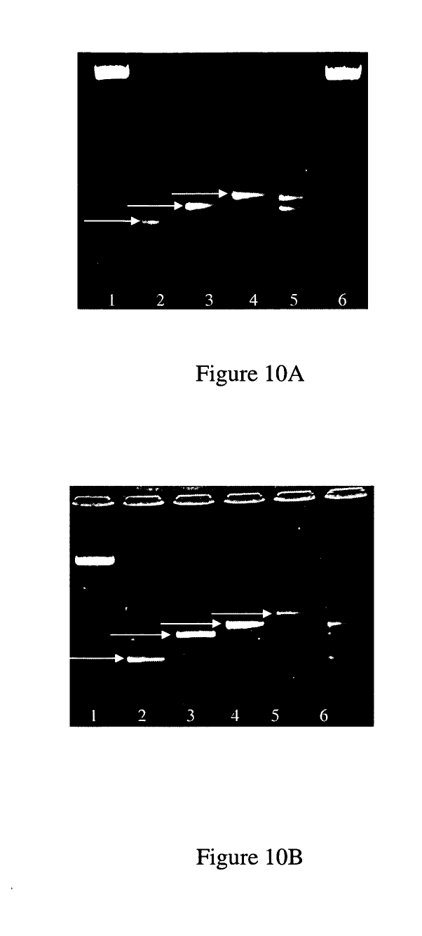

To eliminate unwanted DNA, the ligation mix was treated with nuclease to destroy all linear molecules but preserve circular molecules. The circular DNA was then linearized utilizing Not I restriction enzyme. PCR was then used to amplify all fragments captured in closed circles using primer binding sites in the adapter with Vent or Taq DNA polymerase (New England Biolabs). All three fragments were successfully amplified from E. coli genomic DNA when single adapters were included in the ligation mix. Multiplexing of the adapters in which all three were combined into the one ligation also demonstrated the successful amplification of the three fragments. Band sizes appeared larger than the genomic fragment selected because of the primer and adapter sequences at the ends of the fragments (see FIG. 10A).

To amplify four specific fragments from human genomic DNA of 125, 262, 318, and 499 bp in size, two rounds of selection were used. The first round of capture was performed on human genomic DNA using the four adapters separately or with a pool of four adapters (Adapter A). If it is assumed the genome is fragmented into 6,000,000 fragments by the Type IIs restriction digest, then an adapter that is 1 of 32,000 variants will select about 188 unique fragments. However, other structures are likely to form such as two or more genomic fragments ligated and captured into the adapter circle, but their frequency will be lower.

The amplification of specific fragments from the human genome required two rounds of selection. After the first round PCR with primer set A, the products were digested with Fok I enzyme and the DNA was re-ligated with an adapter (Adapter B) specific for the new overhangs generated in the genomic region of the captured DNA. After ligation, the DNA was digested with Bal-31 nuclease and then amplified with alternate PCR primer binding sites to those used in the first round. When adapters specific for a single fragment were used for both the first round and the second round of selection, a single band was produced for the 125, 262, 318, and 499 bp products. When all four A-adapters were combined for the first step ligation and four B-adapters for the second step ligation, three of the four fragments were visible on a gel (see FIG. 10B).

### Example 2

### Dry Etch Step (RIE) Process

5 nm of chromium is sputtered on a silicon wafer, followed by 20 nm of gold. The gold is the electrode for plating. Next, the wafer is spin coated with 10 to 20 μm polymethylmethacrylate (PMMA), the thickness of which depends on the required nanobar length. Next, approximately 500 nm SiO₂ etch stop is deposited followed by spin coating 2 microns of photoresist. The upper layer of resist is exposed with a hole-array pattern and developed. The pattern is transferred to the etch mask with a dry etch step (dry reactive ion etching or RIE). RIE etching is again used to pattern the polymer. The same etch tool and process is used for both etch steps. The wafer is either used in its entirety or diced into smaller plating units and then plated using the usual, or slightly modified, process. A thin layer of zinc is electroplated as a sacrificial release layer, and then the silver, gold and palladium that make up the nanobar design. After electroplating, the resist is removed with acetone. The nanobars are liberated from the substrate by immersing the wafer in an acetic acid bath with sonication.

### Example 3

### Recovery of the Specific Genomic Fragment of Apo E Containing the Codons for Amino Acids 112 and 158

Apolipoprotein E (Apo E) is an important protein involved in the transport and removal of lipids in the blood. A deficiency of Apo E can result in the premature development of atherosclerosis due to the accumulation of lipids in the blood and vasculature. There are many polymorphisms associated with this protein in humans however there are 3 major isoforms that have been studied extensively; Apo E2, E3 and E4. The major isoform is Apo E3 which is present at a frequency of about 70-80% in the human population, Apo E2 occurs with a frequency of about 5-10% and the frequency of the Apo E4 allele is about 10-15%. The presence of the Apo E2 allele has been demonstrated to be associated with increased plasma triglycerides and with the genetic disorder type III hyperlipoproteinemia. In addition to being associated with an increased risk of developing atherosclerosis, Apo E4 has been shown to be associated with Alzheimers disease and other neurological pathologies. Table 5 shows the major isoforms of Apo E generated through single nucleotide polymorphisms (SNPs) that produce altered amino acids at positions 112 and 158 of the protein, wherein C=cytosine, G=guanine, T=thymine.

TABLE 5

| Apo E | Amino acid 112 | Amino acid 158 |
|---|---|---|
| isoform | (codon) | (codon) |
| Apo E2 | Cysteine (TGC) | Cysteine (TGC) |
| Apo E3 | Cysteine (TGC) | Arginine (CGC) |
| Apo E4 | Arginine (CGC) | Arginine (CGC) |

### A. Testing the Genomic Fractionation Technique on a Bacterial Artificial Chromosome (BAC) Containing the Apo E Gene.

Analysis of publicly available sequence databases such as the UCSC genome browser (Karolchik et al., Nucl. Acids Res. 31:51-54 (2003)) are used to identify BAC clones that contain the 3.7 kilobase Apo E gene. A 153 kilobase BAC clone RP1 1-47010 (Children's Hospital Oakland Research Institute-BACPAC resources) is selected that contains the Apo E gene in addition to many other important apolipoprotein genes such as apo CI and apo CII.

After isolation of the purified BAC by standard plasmid isolation techniques the BAC is digested with a Type IIS restriction enzyme selected from the list in Table 6. The sequence of the BAC is analyzed for the recognition sites of each of the enzymes to assess the restriction patterns and nature of the fragments produced. Table 6 also shows the size of the specific fragment that contains the amino acid 112 polymorphism and the number of fragments obtained by digestion with the enzyme.

Two enzymes are selected from the list that produces a small or large number of fragments to assess the effect on recovery of a unique fragment. SfaN I produces 147 genomic fragments and Bsm AI produces 798 genomic fragments. SfaN I and BsmA I both produce a fragment that contains both codons for amino acid 112 and 158. The BAC clone is digested with each of the enzymes separately and the restriction enzyme digests analyzed by agarose gel electrophoresis to assess the quality of the digests.

TABLE 6

| | | | | |
|---|---|---|---|---|
| | | Amino | | |
| | | acid 112 | Fragment | Average |
| Enzyme | | fragment | number in | fragment |
| name | Recognition sequence and cut site | size | BAC | size |

| | | | | |
|---|---|---|---|---|
| Bbv I | GCAGCNNNNNNNN\hat{ }NNNN_{—} | 108 | 359 | 426 |
| SfaN I | GCATCNNNNN\hat{ }NNNN_{—} | 398 | 147 | 1041 |
| Fok I | GGATGNNNNNNNNN\hat{ }NNNN_{—} | 405 | 354 | 432 |
| BsmF I | GGGACNNNNNNNNNN\hat{ }NNNN_{—} | 1369 | 445 | 343 |
| BsmA I | GTCTCN\hat{ }NNNN_{—} | 708 | 798 | 191 |

After digestion of the BAC the restriction enzyme is heat inactivated and the genomic fragments are ligated to an adapter with complementary overhangs to the fragment that contains the codons 112 and 158. The ligation reaction utilizes T4 DNA ligase

TABLE 7

| Enzyme | 5-prime site 3-prime site |
|---|---|
| SfaN I | \CTGT CTCC---intervening sequence---GCGG\CTCC |
| | GACA\GAGG---intervening sequence---CGCC GAGG\ |
| BsmA I | \CGGA GGAG---intervening sequence---CTGC\AGCG |
| | GCCT\CCTC---intervening sequence---GACG TCGC\ |

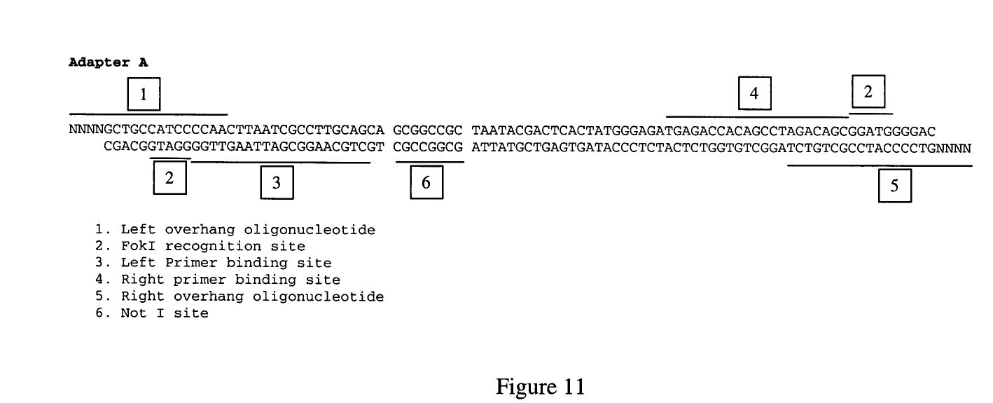

Table 7 describes the nucleotides surrounding the cut sites of SfaN I and BsmA I for the genomic fragment that contains the codons 112 and 158. The sequence of the overhangs for the SfaN I digested genomic DNA is 5′-CTGT and 5′-GGAG. Therefore the adapter for this fragment is designed with overhangs 5′-ACAG and 5′-CTCC respectively (see FIG. 11). There are several ways to make all possible 32,768 adapters without having to synthesize each one individually. Two 62-mer oligonucleotides are synthesized, are heated to 95° C. and then slowly cooled and annealed by virtue of complementary bases.

To produce all 32,768 adapters, 256 oligonucleotides are produced of each strand with all 4 base combinations at the 5-prime end. Alternatively, common core sequences are amplified by PCR and short 8-9 base oligonucleotides are attached at the ends to generate the variable adapter types.

The adapters contain a restriction site for future release of the captured fragment a further 4-bases into the genomic sequence. For example Aar I enzyme is selected because it has a 7-base recognition sequence (so it normally cuts infrequently), and produces a 4-base overhang located four bases away from the recognition sequence (CACCTGCNNNN\hat{ }NNNN_). The adapter also contains primer binding sites for PCR amplification of the fragment.

The ligated adapter and genomic fragment is treated with Lambda exonuclease and Exonuclease I. Lambda exonuclease digests the linear double-stranded DNA from the 5-prime end and Exonuclease I will digests the newly generated single-stranded DNA from the 3-prime end.

After heat inactivation of the nucleases, the circular fragments are amplified by either Rolling Circle Amplification (RCA), or inverse PCR with primers P1 and P2 to assess the most effective method for amplification. The primers for rolling circle amplification are random hexamers because only closed circular DNA are amplified. A portion of the sample is not amplified by PCR or RCA at all, but used directly in the second stage of selection to assess the need for this amplification step. The adapter may include an 8-base restriction enzyme recognition site such as PmeI to prevent the adapter from interfering in the second stage ligation, if RCA is used for amplification.

The linear amplified DNA that is produced from this step is likely to be the fragment that contains the Apo E polymorphism because the probability that 2 fragments would be produced with the same set of 4 base overhangs is 1 in 32,000 and only 147 or 798 fragment types are produced. A second digestion is applied to the PCR amplified DNA using the enzyme Aar I to digest the amplified genomic DNA to generate a fragment with 5′-CTCC and 5′-CCGC overhangs on each end for the SfaN1 original fragment (Table 6). An adapter is prepared with GGAG and GCGG overhangs and ligated to the digested PCR amplification mix. After ligation the sample is digested with Lambda exonuclease and Exonuclease I before amplification with primers to sequences in the adapter resulting in amplification of the 398 (or 708 bp band) base pair band containing the AA112 and 158 polymorphism for the Apo E gene.

### B. The Genomic Fractionation Technique on Human Genomic DNA Containing the Apo E Gene

The amplification of the same fragment from human genomic DNA is done using DNA collected from 1 ml of human blood. Using a commercially available genomic DNA isolation kit up to 20 μg of DNA can be retrieved from 1 ml of blood. The DNA is digested with SfaN I and BsmA I as described for the BAC. For a 200 bp fragment this represents about 1 pg of DNA for that fragment. Ligation of the specific adapters is done as performed with the BAC. The closed circular DNA is amplified by Inverse PCR (or RCA) separately. A portion of the sample is not amplified but used directly in the second stage of selection.

1. A method of amplifying genomic fragments, comprising the steps:
(a) digesting genomic DNA into genomic fragments, wherein said digesting results in genomic fragment overhangs;
(b) contacting said genomic fragments with one or more adapters, wherein said adapters are complementary to at least two of said overhangs;
(c) ligating said adapters to said genomic fragment overhangs to form closed adapter-genomic fragment circles;
(d) separating said adapter-genomic fragment circles from linear fragments; and
(e) amplifying said adapter-genomic fragment circles.

2. A method of amplifying genomic fragments, comprising the steps:
(a) digesting genomic DNA into genomic fragments, wherein said digesting results in genomic fragment overhangs;
(b) contacting said genomic fragments with one or more adapters, wherein said adapters are complementary to at least two of said overhangs;
(c) ligating said adapters to said genomic fragment overhangs to form closed adapter-genomic fragment circles;
(d) modifying said circles by cutting with one or more restriction enzymes binding to one or more adapter sites; and
(e) amplifying said adapter-genomic fragment circles.

3. The method of claim 1 or 2, wherein said digestion is performed by a Type IIS enzyme.

4. The method of claim 2 wherein said restriction enzyme is a Type IIS enzyme.

5. The method of claim 3 or 4, wherein said Type IIS enzyme is selected from the consisting of Bbv I, SfaN 1, Fok I, BsmF I, and BsmA I.

6. The method of claim 3, wherein said enzyme has a specific recognition site and a separate digestion site.

7. The method of claim 5, wherein said enzyme has a four-, five-, or six-base recognition sequence.

8. The method of claim 1 or 2, wherein said overhangs are two, three or four bases long.

9. The method of claim 1 or 2, wherein said amplification is selected from the group consisting of rolling circle PCR and inverse PCR.

10. The method of claim 1 or 2, wherein said adapters comprise one or two pairs of Type IIS restriction enzyme recognition sites, each pair positioned at one adapter end for cutting into the genomic and adapter DNA thereby forming matching overhang sequences.

11. The method of claim 1 or 2, wherein uracil is incorporated into the adapter sequence.

12. The method of claim 12, wherein the adapter is treated with uracil-DNA glycosylase prior to amplification.

13. The method of claim 1, wherein said separating is performed by digesting said linear fragments with an enzyme.

14. The method of claim 13, wherein said enzyme is an exonuclease.

15. The method of claim 1, wherein said separating is performed by biotinylated blocking adaptors and streptavin-coated beads combined with magnetic attraction or centrifugation.

16. The method of claim 15, wherein said biotinylated blocking adapters comprise a first and a second end, said first end having an overhang complimentary to said overhangs of said universal adapters, and said second end covalently bound to at least one biotin molecule.

17. The method of claims 1 or 2, wherein at least one blocking adapter is included in the ligating step.

18. Two sets of universal building blocks comprising:
a first set of single-stranded oligonucleotides having a first end and a second end, said first end having a sticky-end overhang and said second end having sequence of 8-20 bases; and a second set of single-stranded oligonucleotides having a first end and a second end, said first end having a sticky-end overhang and said second end having a sequence of 8-20 bases, wherein said first ends of said first and second sets are different, and said second end of said first set are complementary to said second end of said second set, generating all possible combinations of adapter sequences.

19. The sets of universal building blocks of claim 18, wherein said first and second set are comprised of up to 64 different first end 3-base overhangs.

20. The sets of universal building blocks of claim 18, wherein said first and second set are comprised of up to 256 different first end 4-base overhangs.

21. The sets of universal building blocks of claim 18, wherein said first and second set are comprised of up to 1024 different first end 5-base overhangs.
